(19) United States
(12) Patent Application Publication
(10) Pub. No.: US 2005/0155087 A1
(43) Pub. Date: Jul. 14, 2005

(54) Method of screening compounds

(76) Inventors: Leonard I. Zon, Wellesley, MA (US); Howard M. Stern, Newton, MA (US); Ryan Murphey, Brookline, MA (US)

Correspondence Address: DAVID S. RESNICK, 100 SUMMER STREET, NIXON PEABODY LLP, BOSTON, MA, 02110-2131, US

(21) Appl. No.: 10/499,234

(22) PCT Filed: Dec. 17, 2002
(86) PCT No.: PCT/US02/40262

Related U.S. Application Data
(60) Provisional application No. 60/341,428, filed on Dec. 17, 2001.

(51) Int. Cl. A01K 67/027; A61K 49/00

(52) U.S. Cl. 800/3; 800/20

(57) ABSTRACT

The present invention is directed to a novel, target-blind approach to drug discovery. The concept is to model human phenotypes in a teleost, such as a zebrafish, and then screen compounds, e.g., small molecules, for their ability to alter the phenotype. Because the screen is performed with a whole vertebrate organism and uses a phenotype as the output, the need to first identify target genes is eliminated. This approach is powerful because a single screen can theoretically detect drugs affecting any target relevant to the phenotype being observed, even if those targets are not yet characterized.

### BACKGROUND OF THE INVENTION

The traditional approach to drug discovery is to identify target genes involved in a disease and then design an in vitro assay to screen small molecules for alterations in function of the target. The traditional approach is flawed not only with the high cost and inefficacy due to the animal models available and the time expenditure involved in identifying target genes, but also with the fact that the protein configurations used in most pharmaceutical industry assay systems (the protein is typically in crystalline form, in simple aqueous solution, and attached to a fixed bed or overexpressed in a transfected cell) are radically different from the in vivo state. Horrobin D. F., Realism in drug discovery—could Cassandra be right? Nature Biotech. 19, 1099-1100 (2001). Thus, a system which is less costly and more efficient, and wherein the targets are found in their native configuration is desired.

Both mice and Drosophila have proven to be powerful models for determining which genes are important in the development of human phenotypes, including disease phenotypes such as cancer. Mice are particularly useful for reverse genetics in which genes of interest are overexpressed or deleted followed by phenotypic analysis. For example, many tumor suppressor genes and oncogenes have been studied by these approaches, and the cancers that develop in these mice histologically resemble human neoplasms. McClatchey, A. and T. Jacks, Tumor suppressor mutations in mice: the next generation. Curr. Opin. Genet. Develp. 8, 304-310 (1998); Eva, A., Use of transgenic mice in the study of proto-oncogene functions. Semin. Cell Bio. 3, 137-145 (1992). However, forward genetic screens for recessive mutations in mice are difficult due to high cost and tremendous space requirements. In addition, whole embryo-based small molecule screens are not practical.

Drosophila is a powerful organism for forward genetic screens. For example, various genetic screens have identified more than 50 genes which when mutated cause hyperplastic or neoplastic growth. Watson, K. L., R. W. Justice, and P. J. Bryant, Drosophila in cancer research: the first fifty tumor suppressor genes. Cell Sci. Suppl. 18, 19-33 (1994). Some of these genes have proven to be relevant to mammalian neoplasia. For example, the gene large tumor suppressor (LATS) when deleted in mice results in soft tissue sarcomas and ovarian tumors. Mechler, B. M., W. McGinnis, and W. J. Gehring, Molecular cloning of lethal(2)giant larvae, a recessive oncogene of Drosophila melanogaster. EMBO J. 4, 1551-1557 (1985); St. John, M. A., W. Tao, X. Fei, R. Fukumoto, M. L. Carcangiu, D. G. Brownstein, A. F. Parlow, J. McGrath, and T. Xu, Mice deficient of Latsl develop soft-tissue sarcomas, ovarian tumours and pituitary dysfunction. Nat. Genet. 21, 182-186 (1999). However, the neoplasias seen in Drosophila do not histologically resemble mammalian neoplasms, nor do they exhibit malignant behavior (i.e. metastasis). In addition, as with mice, Drosophila are not readily compatible with whole organism-based small molecule approaches.

Therefore, due to inefficacies and cost associated with the traditional approaches to drug discovery and due to difficulties associated with handling proteins in vitro, there remains a need for improved methods to drug discovery.

### SUMMARY OF THE INVENTION

The present invention is directed to a novel, target-blind approach to drug discovery. The concept is to model human phenotypes, for example disease phenotypes, in a teleost such as a zebrafish and then screen compounds, e.g., small molecules, for their ability to alter the phenotype. Because the screen is performed with a whole vertebrate organism and uses a phenotype as the output, the need to first identify target genes is eliminated. This approach is very powerful because a single screen can theoretically detect, for example, drugs affecting any target relevant to a disease phenotype being observed, even if those targets are not yet characterized.

In one aspect, the present invention is directed to a method of screening a test compound for the ability of the compound to alter a phenotype which resembles a human phenotype. The method comprises the steps of (a) contacting at least one teleost that has inherited the phenotype with a test compound, and (b) detecting the teleost from step (a) in which the phenotype is altered. The term “change” is meant to indicate an alteration in the inherited phenotype of a teleost. A chemical compound is considered to change the phenotype when the statistically expected pattern of phenotype inheritance is skewed towards fewer mutants than expected in the presence of a test compound. For example, a change can be detected in embryos, wherein the embryos are produced by mating heterozygous zebrafish which have a lethal recessive phenotype with each other. The resulting embryos are consequently contacted with a test compound, as explained in detail in the examples below, and visually examined for, for example, increased or decreased P-H3 staining under a light microscope. A chemical compound is considered to change the phenotype if greater than about 75%, and most preferably about 95% of the embryos contacted with the test compound exhibit a wild-type phenotype pattern, for example a wild-type P-H3 staining pattern.

The “observable” phenotype observed depends on the teleost model used and includes any observable physical or biochemical characteristic of the teleost. The phenotype can be associated with, for example, organ development, protein phosphorylation status, mitotic spindle formation, protein expression, cell morphology, or a disease in general. The phenotype can be, for example, a morphological change, a change in gene expression, a change in tumor formation susceptibility. In general, the phenotype change can be observed using various suitable means including microscopy with or without immunohistochemical staining and RNA-quantification.

For example, in a cancer model one could look for changes versus the wild type, i.e., alteration of cell cycle proteins or phosphorylation status of cell cycle proteins. In the preferred embodiment of the method of the present invention, the phenotype is characterized by phosphorylated or dephosphorylated cell cycle protein.

In one embodiment, the phenotype is a disease phenotype. The disease phenotype contemplated by the method of the present invention is associated with, among others, cancer, hematologic disease, immunologic disease, angiogenesis, bone diseases, cardiovascular disease, obesity, diabetes, or neurodegenerative disease.

The term “teleost” as used herein means of or belonging to the Telostei or Teleostomi, a group consisting of numerous fishes having bony skeletons and rayed fins. Teleosts include, for example, zebrafish, medaka, Giant rerio, and puffer fish. In one embodiment of the invention, the teleost is a zebrafish. The teleost can be an embryo, larva or adult. In certain preferred embodiments, the teleost is a zebrafish embryo.

In one embodiment of the present invention, the teleost can be contained in an aqueous medium in a microtiter well.

In another embodiment of the present invention, the test compound is administered to the teleost by dissolving the test compound in media containing the teleost.

The term “test compound” as used herein comprises any element, compound, or entity, including, but not limited to, e.g., a pharmaceutical, a therapeutic, a pharmacologic, an environmental or an agricultural pollutant or compound, an aquatic pollutant, a cosmetic product, a drug, a toxin, a natural product, a synthetic compound, or a chemical compound or a mixture thereof which can be mixed with, or alternatively, dissolved in an aqueous mixture. The test compound can further include nucleic acids, peptides, proteins, glycoprotein, carbohydrates, lipids, or glycolipids and mixtures thereof. The test compounds that are shown to alter the teleost phenotype, for example, a disease phenotype, can then be further tested in other animal disease models.

In the method of the present invention, the test compound is administered to the teleost by injecting the test compound into the teleost or is administered in conjunction with a carrier. The carrier can be a solvent, lipid or peptide.

In the method of the present invention more than one test compound can be screened simultaneously or sequentially.

The method comprises (a) contacting a teleost having a phenotype with a test compound in varying concentrations, and (b) detecting or observing whether there is an alteration in the phenotype in the teleost of step (a), wherein an alteration detected in step (b) indicates that the test compound is effective.

In yet another aspect, the present invention provides a method of screening a test compound for the ability of the compound to alter a cell-cycle associated phenotype. The method comprises contacting at least one wild type teleost with a test compound and detecting the teleost in which the phenotype is altered. The preferred phenotype is cell-cycle associated protein expression or cell-cycle associated protein phosphorylation status. Example of cell-cycle associated proteins include, but are not limited to histone H3, MAP kinase, MEK-1, BM28, cyclin E, p53, Rb and PCNA.

The present invention also includes a compound obtained by the screening methods outlined above.

The present invention further includes a method of treating a subject in need thereof, such as human, having a cell cycle defect phenotype, comprising administering to the subject a compound obtained by the screening methods outlined above. A detectable cell cycle defect phenotype includes, but is not limited to, cancer.

Unless otherwise defined, all technical and scientific terms used herein have the same meaning as commonly understood by one of ordinary skill in the art. Although methods and materials similar or equivalent to those described herein can be used in the practice or testing of the invention, the preferred methods and materials are described below. All publications, patent applications, patents and other references mentioned herein are incorporated by reference. In addition, the materials, methods and examples are illustrative only and not intended to be limiting. In case of conflict, the present specification, including definitions, controls.

### BRIEF DESCRIPTION OF THE FIGURES

The accompanying figures, which are incorporated in and constitute a part of this specification, illustrate embodiments of the invention and, together with the description, serve to explain the objects, advantages, and principles of the invention. In the figures:

FIG. 1 illustrates crash & burn tumor incidence by genotype (+/+=wildtype; +/−=heterozygote).

FIG. 2 is a schematic of one embodiment of the screening system contemplated by the present invention.

FIG. 3 illustrates a matrix pooling strategy. To improve screening efficiency, matrix pooling may be performed. For example, 16 chemicals can be pooled horizontally and vertically, generating 8 pools of 4 (letters). Thus, the number of wells that need to be scored is cut in half. A compound is considered a “hit” only if phenotype appears in one horizontal pool and one vertical pool. The intersection of the pools in the grid identifies the compound of interest (gray). This method is most effective if the hit rate and the toxicity rate are both low.

FIGS. 4A-4C show that 8G16 prevents the phenotypic appearance of crash & burn although the genotype still reflects the crash & burn (crb) mutation. FIG. 4A shows an untreated wild-type zebrafish embryo, FIG. 4B shows an untreated crash & burn mutant zebrafish embryo and FIG. 4C shows a crash & burn embryo treated with 10 μM 8G16. The staining is with P-H3 antibody and is shown as black dots.

FIGS. 5A-5C show the accumulation of cell in the G1/S phase of the cell cycle when the zebrafish embryos are treated with 8G16. FIG. 5A shows P-H3 staining of an untreated embryo, FIG. 5B shows P-H3 staining of an embryo treated with 100 μM 8G16. FIG. 5C is a FACS analysis of the cell cycle from the cells of the untreated embryos, (control), note particularly the peak on the right hand side, and embryos treated with 10 and 100 μM 8G16.

FIGS. 6A-6E show the ability of 8G16 to rescue a crash&burn (crb) zebrafish mutant (FIGS. 6B, untreated crb mutant and 6D 8G16 treated crb mutant) but not another polyploid zebrafish mutant cds (FIGS. 6C, untreated cds mutant and 6E, 8G16 treated cds mutant) compared to a wild-type, untreated embryo (FIG. 6A).

FIGS. 7A-7C show examples of untreated embryos (FIG. 7A), embryos treated with group II compounds in FIG. 11C, (FIG. 7B, decreased P-H3 staining) and embryos treated with group III compounds (FIG. 7C, increased P-H3 staining).

FIGS. 8A-8B illustrate the structure of 8G16 (FIG. 8A) and inactive compounds that share structural homology with 8G16 (FIG. 8B).

FIGS. 9A-9C illustrates the structure activity relationships of compounds A and L. FIG. 9A shows compounds L1, L2 and L8 which show no activity. FIG. 9B shows compounds L, L4, L5 and A which have similar activity as L. FIG. 9C shows compounds L3, L7, L9, and L10 which show activity only at 5-fold higher concentrations compared to compounds in FIG. 9B.

FIGS. 10A-10C show compounds which share partial homology to the compound in FIG. 10A and result in different results. Compounds in FIG. 10B result in mitotic arrest at concentrations indicated above the compound and compounds in FIG. 10C result in no mitotic arrest in concentrations tested up to 1.5 mM.

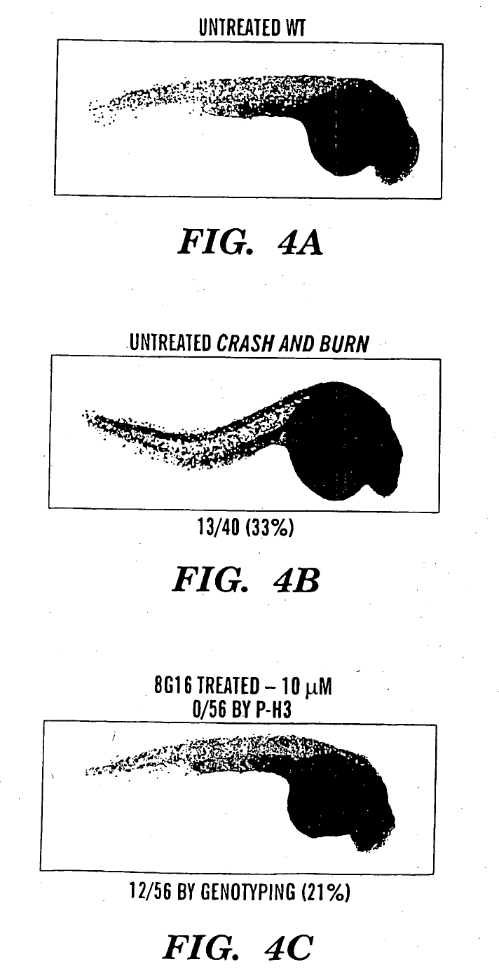

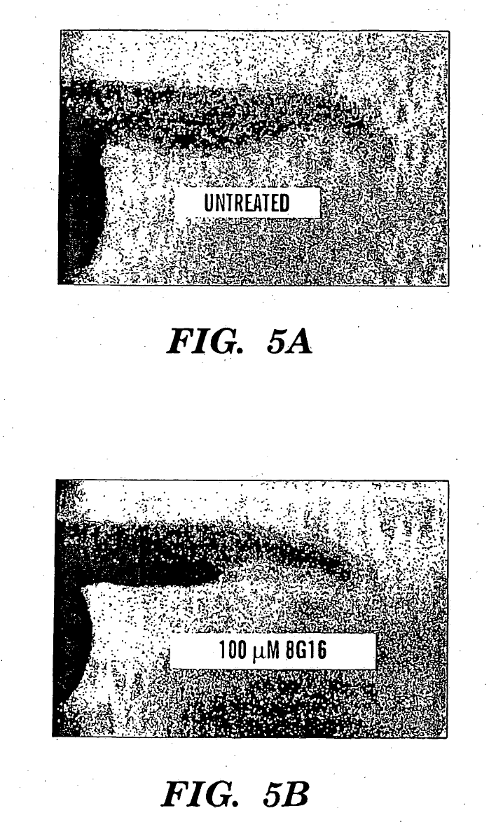

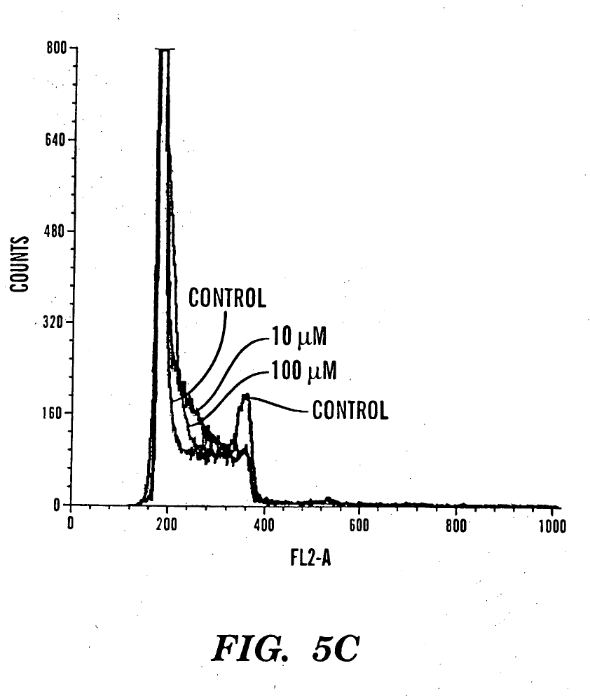

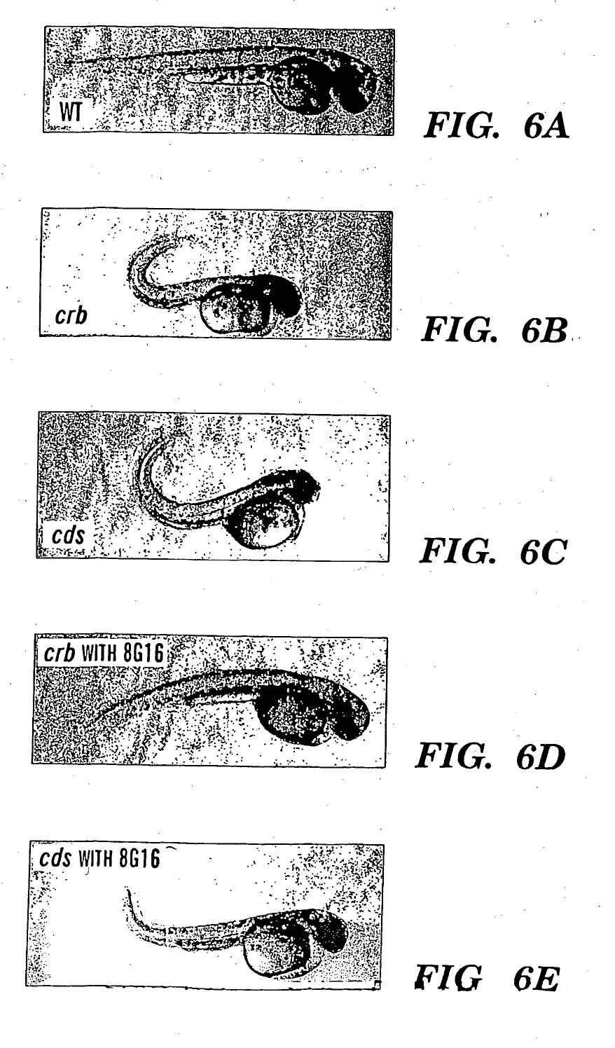

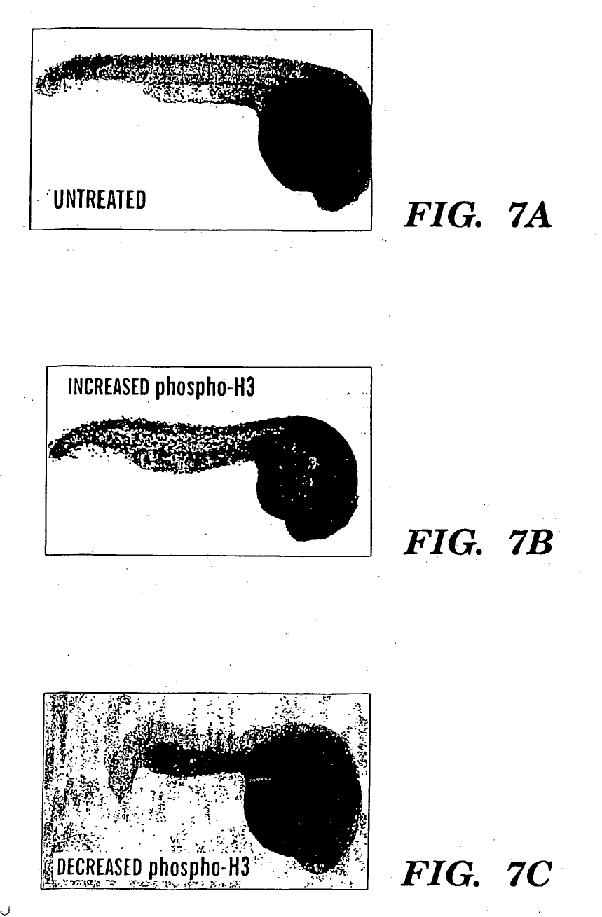

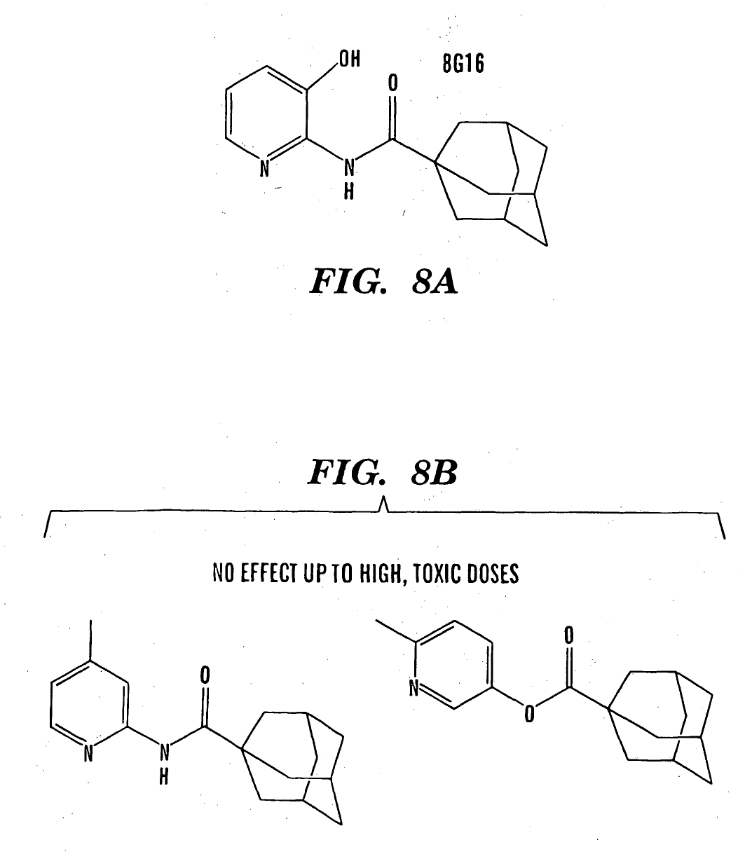

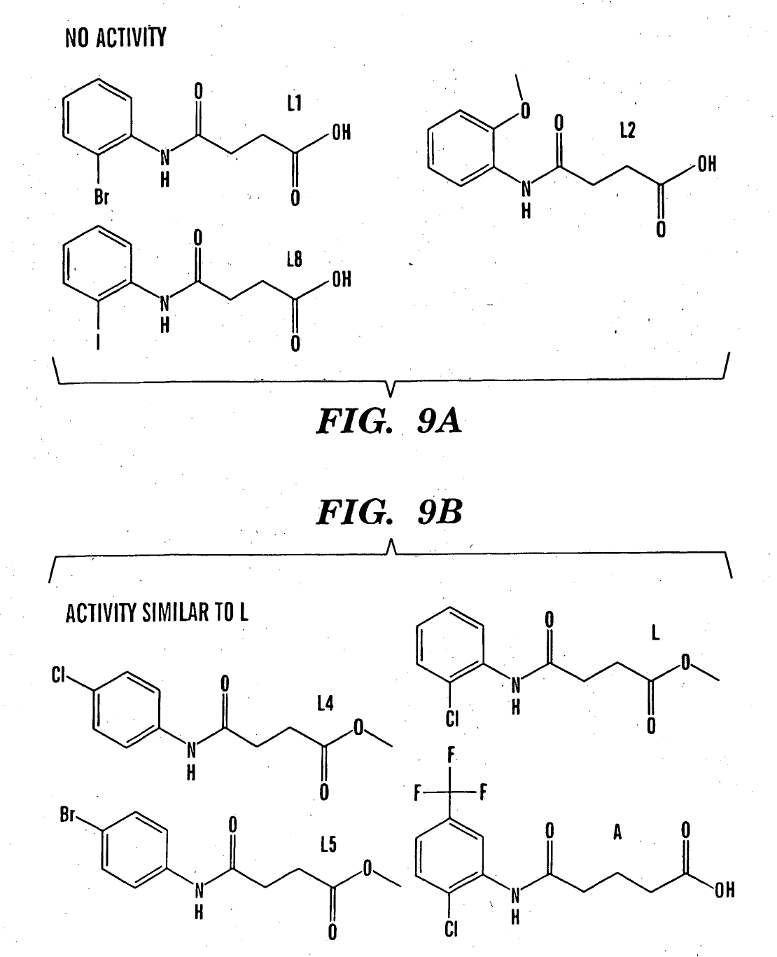

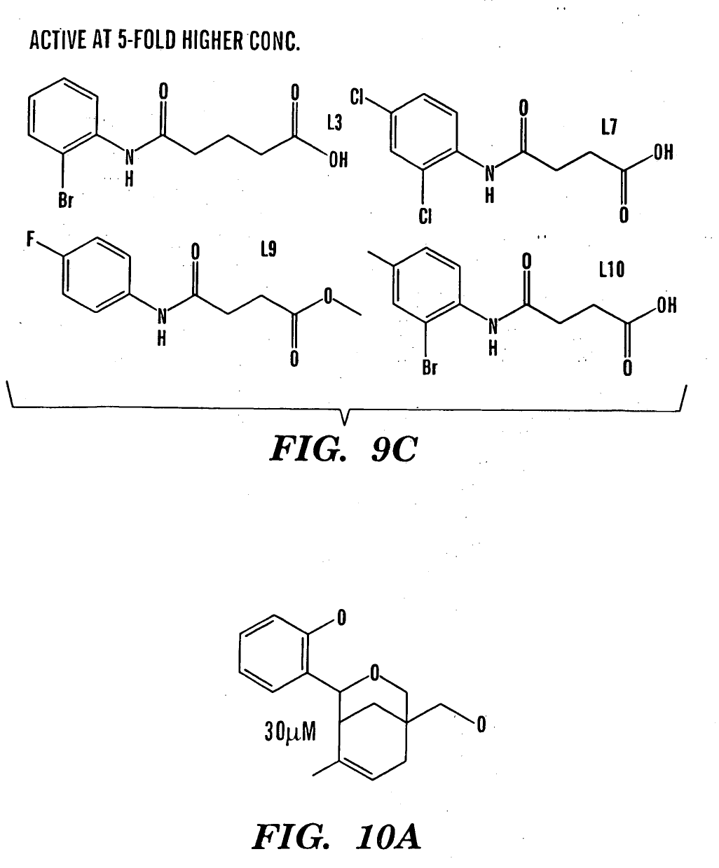

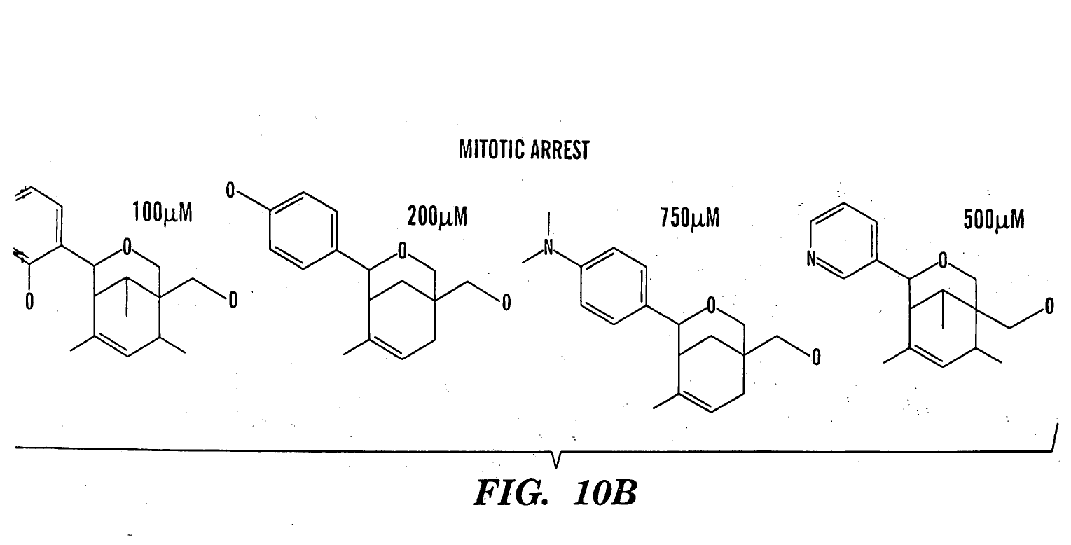

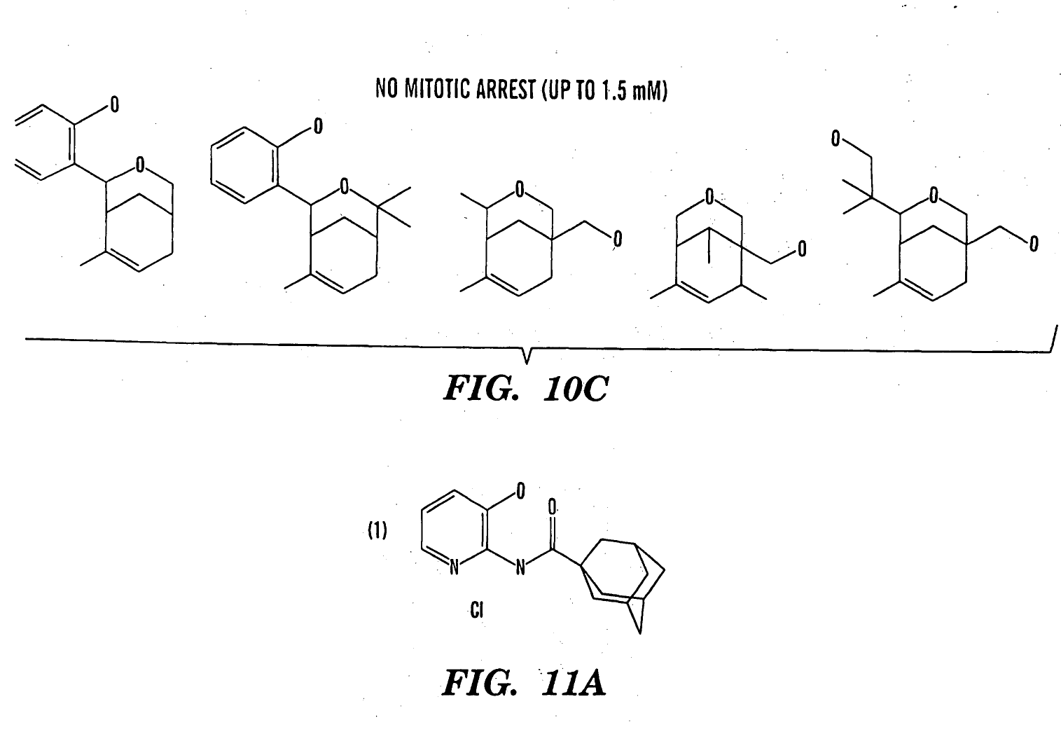

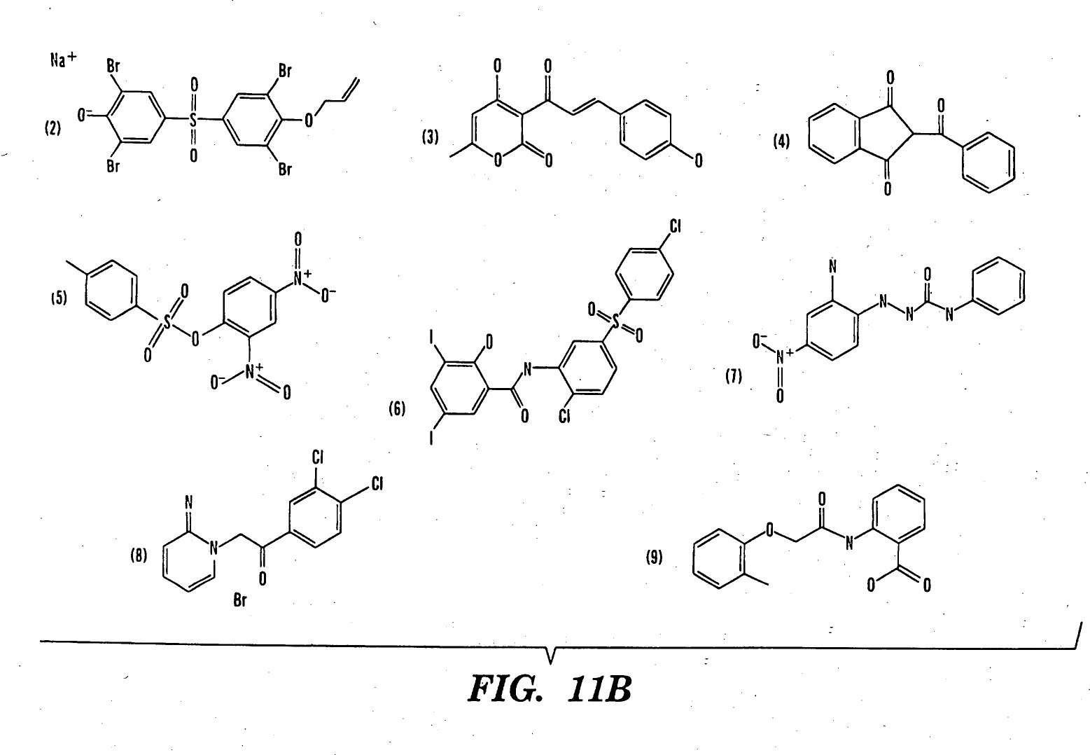

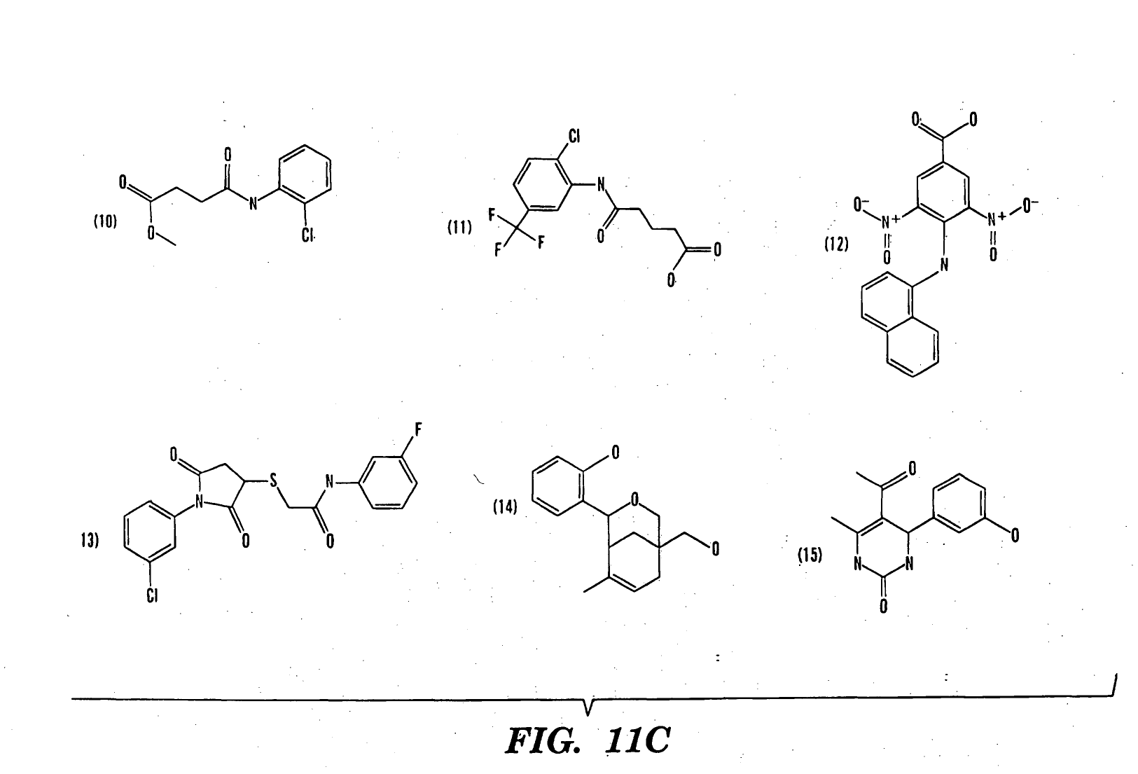

FIGS. 11A-11C show examples of chemical compound structures having an effect when administered to zebrafish mutants. FIG. 11A shows that “8G16” (Group 1, compound number (1)) prevents the crash and burn cell cycle phenotype through 24 hours of development without affecting normal embryos. This compound, Adamantane-1-carboxylic acid (3-hydroxy-pyridin-2-yl)-amide, is a candidate agent for cancer chemotherapy and/or chemoprevention. FIG. 11B shows eight compounds (2-9), Group II including:
(2) 4-(4-Allyloxy-3,5-dibromo-benzenesulfonyl)-2,6-dibromo-phenol
(3) 4-Hydroxy-3-[3-(4-hydroxy-phenyl)-acryloyl]-6-methyl-pyran-2-one
(4) 2-Benzoyl-3a,7a-dihydro-indene-1,3-dione
(5) Toluene-4-sulfonic acid 2,4-dinitro-phenyl esier
(6) 3,5-Diiodo-N-[2-chloro-5-(4-chloro-benzenesulfonyl)-phenyl]-2-hydroxy-benzamide
(7) 1-(2-Amino-4-nitro-phenylamino)-3-phenyl-urea
(8) 1-(3,4-Dichloro-phenyl)-2-(2-imino-2H-pyridin-1-yl)-ethanone
(9) 2-(2-o-Tolyloxy-acetylamino)-benzoic acid that decrease P-H3 staining in crash and burn embryos and also decrease staining in wildtype embryos. Therefore, they likely arrest the cell cycle in G1, S or early G2 when histone H3 is not phosphorylaied. This activity is analogous to the activity of aphidicolin in FIG. 4. These compounds are candidate agents for cancer chemotherapy. FIG. 11C shows Group III compounds. These 6 compounds (10-15) increase P-H3 staining in wildtype embryos and include
(10) N-(2-Chloro-phenyl)-succinamic acid methyl ester
(11) 4-(2-Chloro-5-trifluoromethyl-phenylcarbamoyl)-butyric acid
(12) 4-(Naphthalen-1-ylamino)-3,5-dinitro-benzoic acid
(13) 2-[1-(3-Chloro-phenyl)-2,5-dioxo-pyrrolidin-3-ylsulfanyl]-N-(3-fluoro-phenyl)-acetamide
(14) 2-(5-Hydroxymethyl-8-methyl-3-oxa-bicyclo[3.3.1]non-7-en-2-yl)-phenol
(15) 5-Acetyl-4-(3-hydroxy-phenyl)-6-methyl-3,4-dihydro-1H-pyrimidin-2-one.

Based on screens of the same library by other investigators at the ICCB, these results represent novel activities for these chemicals. These compounds are candidates for cancer chemotherapy.

### DETAILED DESCRIPTION

The present invention provides a novel, target-blind approach to drug discovery, wherein human phenotypes are modeled in a teleost such as a zebrafish and compounds, e.g., small molecules, are screened for their ability to alter the phenotype.

In one aspect, the present invention is directed to a method of screening a test compound for the ability of the compound to alter a teleost phenotype. In the preferred embodiment the phenotype is a disease phenotype. The method comprises the steps of (a) contacting at least one teleost that has an observable phenotype with a test compound, and (b) detecting the teleost from step (a) in which a change in the phenotype indicates a compound capable of altering said phenotype.

The methods of the present invention are generally applicable for use in a teleost. Suitable teleosts include, for example, zebrafish (Danio rerio), Medaka, Giant rerio, and puffer fish. Zebrafish are preferred. Depending on the model used, the zebrafish can be an embryo, larva or adult. Most preferably, for certain embodiments a zebrafish embryo is used.

The disease phenotype contemplated by the method of the present invention is associated with, among others, cancer, hematologic disease, immunologic disease, angiogenesis, bone diseases, cardiovascular disease, obesity, diabetes, or neurodegenerative disease.

Cancer: A number of markers can be used to screen for zebrafish cell cycle mutants and to characterize identified mutants, various cell cycle markers can be examined for the ability to stain proliferating cells in whole zebrafish embryos. Several antibodies that bind to mammalian cell cycle proteins, including phosphorlylated histone H3, phosphorylated MAP kinase, phosphorylated MEK-1, BM28, cyclin E, p53, Rb and PCNA, can be used on whole zebrafish embryos at 12 to 48 hours of development.

For example, a polyclonal antibody directed against the phosphorylated serine 10 residue of histone H13 stained cells in specific embryonic mitotic domains at appropriate times in development. For example, there is high pH3 staining in the eye and developing nervous system at 24 to 36 hours post-fertilization (hpf) when these tissues are known to be highly mitotically active. In the eye, regions with pH3-positive cells are distinct from domains where cell death is occurring. Phospho-H3 staining was appropriately absent from cells that exited the cell cycle as a result of ionizing radiation. The number of pH3 stained cells in irradiated embryos reached a nadir 30 min. post-irradiation and then gradually recovered normal staining by about 2 hours.

Thus, in a preferred embodiment, an anti-pH3 antibody is used as a cell cycle marker in zebrafish using the method of the present invention. An embryonic cell cycle defect can be primary (e.g. mutation in a CDK) or could be secondary (e.g. mutation in a gene involved in DNA repair or replication causing checkpoint activation). Without wishing to be bound by theory, we believe that the embryonic cell cycle defect will correlate with increased cancer prevalence in adults. For example, a preliminary haploid ethylnitrosourea (ENU) mutagenesis screen for altered pH3 staining has been performed. ENU mutagenized male fish (WIK strain) were mated to wild-type WIK females. The F1 offspring were raised to adulthood and F1 females were squeezed to collect their eggs. These clutches were then fertilized with UV irradiated sperm, creating haploid embryos. At 36 hours of development, the clutches were fixed in paraformaldehyde (PFA) and were immunostained with a pH3 antibody. Because the clutches are haploid, any given mutation should affect half of the embryos. Of 750 F1 females that have been screened, clutches from 41 exhibited altered pH3 staining in 50% of the clutch. 21 of these had increased numbers of pH3-positive cells, 11 had decreased numbers of pH3-positive cells, and 9 had other phenotypes such as larger appearing nuclei (stained by pH3). The 41 F1 females carrying the putative mutations of interest were mated to wild-type WIK males, and the F2 offspring were raised to adulthood for re-identification of heterozygote pairs. Half of the F2 generation should be heterozygous for the mutation, thus for each putative mutant, multiple (at least 20) random F2 sibling intercrosses (incrosses) were performed. The F3 embryos were pH3 stained at 36 hours to determine if the mutation had been recovered in the diploid fish. Seven mutants have been recovered from the 41 F1 females.

Phenotypes associated with cancer include, for example, changes in gene expression compared to a wildtype or normal fish of cell cycle proteins or phosphorylation status of the cell cycle proteins. Examples of such models are discussed below in the Examples. Methods for producing those models and others are disclosed in U.S. application Ser. No. 09/758,007 filed Jan. 10, 2001, the content of which is incorporated herein by reference.

Hematologic diseases: There are over 50 zebrafish mutations which have been identified to affect blood cell development. Ransom, D. G. et al., Characterization of zebrafish mutants with defects in embryonic hematopoiesis. Developmnent 123, 311-319 (1996); Weinstein, B. M. et al., ematopoietic mutations in the zebrafish. Development 123, 303-309 (1996). These mutations can be grouped into stem cell mutants, blood cell differentiation/proliferation mutants, hypochromic mutants and photosensitive mutants. The stem cell and blood cell differentiation/proliferation mutants are excellent model systems for various forms of human anemias. Brownlie, A., A. Donovan, S. J. Pratt, B. H. Paw, A. C. Oates, C. Brugnara, H. E. Witkowska, S. Sassa, and L. I. Zon, Positional cloning of the zebrafish sauternes gene: a model for congenital sideroblastic anemia. Nat. Genet. 20, 244-250 (1998). The hypochromic mutants are models for human hemoglobinopathies and for defects in iron transport such as hemochromatosis. Donovan, A. et al., Positional cloning of zebrafish Ferroportin 1 identifies a conserved vertebrate iron exporter. Nature 403, 776-781 (2000). The photosensitive mutants are models for human porphyria. Ransom, D. G. et al., Characterization of zebrafish mutants with defects in embryonic hemaiopoiesis. Development 123, 311-319 (1996). The blood cell phenotype is easily scored by visual inspection of the embryos between 1 and 5 days of development using a dissecting microscope. See Id. For example, O-dianisodine staining can be used as a marker for the presence of heme. See Id. Porphyria phenotypes can be observed by looking for autofluorescence of red cells under ultraviolet light using a dissecting microscope. See Id. Finally, for example, in situ hybridization can be used to track RNA expression of blood genes such as GATA-1, GATA-2, and hemoglobin. See Id. RNA amount can also be observed using numerous different RT-PCR-based RNA quantification methods. These methods are routine to one skilled in the art and include, methods for transcript detection and quantification include Northern-blot hybridization, ribonuclease protection assay, and reverse transcriptase polymerase chain reaction (RT-PCR) based methods. The quantitative RT-PCR based methods useful according to the present invention include, but are not limited to RNA quantification using PCR and complementary DNA (cDNA) arrays (Shalon et al., Genome Research 6(7): 639-45, 1996; Bernard et al., Nucleic Acids Research 24(8): 1435-42, 1996), solid-phase mini-sequencing technique, which is based upon a primer extension reaction (U.S. Pat. No. 6,013,431, Suomalainen et al. Mol. Biotechnol. June; 15(2): 123-31, 2000), ion-pair high-performance liquid chromatography (Doris et al. J. Chromatogr. A May 8;806(1): 47-60, 1998), and 5′ nuclease assay or real-time RT-PCR (Holland et al. Proc Natl Acad Sci USA 88: 7276-7280, 1991).

Drug candidates for the above hematologic disorders can be identified using the methods of the present invention and an appropriate marker of the phenotype (visual inspection of blood cells for stem cell defects and anemia, o-dianisodine for hypochromia, and visual inspectionof autofluorescence for porphyria).

Immunologic disorders: A genetic screen has been performed to identify zebrafish T-cell mutants by screening for alteration of embryonic Rag-1 expression, a marker of T lymphocytes. Trede, N. S., Zon, L. I., Development of T-cells during fish embryogenesis. Dev. Comp. Immunol. 253-263 (1998); Trede, N. S., A. Zapata, and L. I. Zon, Fishing for lymphoid genes. Trends Immunol 22, 302-307 (2001). Mutants with defects in T-cell development may be models for human immunodeficiency. The methods described in the present invention can be used to screen for compounds that, for example, improve thymic Rag-1 expression in the T-cell mutants. One method of detecting changes in the Rag-1 expression is using in situ hybridization with a Rag-1 probe.

Ongoing genetic screens in zebrafish are seeking to identify mutants with defects in myelopoiesis. Bennett, C. M. et al., Myelopoiesis in the zebrafish, Danio rerio. Blood 98, 643-51 (2001). Such myelopoietic mutants can be used as a model for human granulocytic disorders. The chemical suppressor screen of the present invention is useful in identification of lead compounds for such disorders. Therefore, in one embodiment, the invention provides a method of detecting improvement/increase of expression of myeloid markers such as myeloperoxidase or Pu.1 in the presence of test compounds.

Cardiovascular disease: Numerous zebrafish mutants with defects in cardiovascular form and function have been described. Stanier, D. Y. R. et al., Mutations affecting the formation and function of the cardiovascular system in the zebrafish embryo. Development 123, 285-292 (1996); Chen, J. N., Mutations affecting the cardiovascular system and other internal organs in zebrafish. Development 123, 293-302 (1996). Cardiovascular function and morphology can be evaluated visually using a dissecting microscope within the first 5 days of development. Using the methods of the present invention, test compounds can be screened for the ability to improve either cardiac function (e.g. heart rate or wall motion) or morphology in these mutants. Compounds or chemicals having capacity to improve either the cardiac function or morphology identified using the methods of the present invention have potential for treating cardiac failure in adults, boosting cardiac function in children with cardiac developmental defects and preventing a cardiac developmental defect in fetuses at high risk based on genetic predisposition.

Angiogenesis: Zebrafish mutants with defects in vasculogenesis, such as cloche, can further be used in a zebrafish chemical suppressor screen of the present invention to identify chemicals or compounds that stimulate angiogenesis. Stanier, D. Y. R. et al., Mutations affecting the formation and function of the cardiovascular system in the zebrafish embryo. Development 123, 285-292. (1996). Angiogenesis can simply be observed through the transparent embryo or, alternatively, can be detected via in situ hybridization with a vascular marker, for example, a flk-1 probe. Chemicals or compounds that stimulate anglogenesis in the method of the present invention can be useful in development of treatments for, for example, human ischemic disorders. An example would be in cases of myocardial infarction where stimulating myocardial blood vessel development may improve the health of the remaining myocardium. Chiu, R. C., Therapeutic cardiac angiogenesis and myogenesis: the promises and challenges on a new frontier. J. Thorac Cardiovasc Surg 122, 963-971 (2001).

Neurodegenerative diseases: There are numerous zebrafish mutants that exhibit neuronal survival defects. In the method of the present invention, these mutants can be used as models of neurodegenerative disorders in humans. One could screen for, for example, chemical suppressors of neuronal cell apoptosis using acridine orange staining or TUNEL staining to identify DNA fragmentation. Abdelilah, S. et al., Mutations affecting neural survival in the zebrafish, Danio rerio. Development 123, 217-227 (1996); Furutani-Seiki, M., Neural degeneration mutants in the zebrafish, Danio rerio. Development 123, 229-239 (1996).

Bone diseases: Ongoing zebrafish genetic screens are finding bone development mutants that are models for human diseases. The laboratory of Shannon Fisher at John Hopkins University has found a zebrafish model of osteogenesis imperfecta. Screening fish using roentgenograms serves to identify fish with abnormal bone density. Fish with altered bone density could be models not only for genetic disorders such as osteogenesis imperfecta, but could also be models for adult diseases such as osteoporosis and osteopetrosis. A chemical suppressor screen could be performed on young fish by taking roentgenograms and looking for improvements in bone density in chemical treated fish.

Diabetes: Several zebrafish mutant have defects in pancreatic islet development. For example, floating head mutants develop only small remnants of endocrine pancreas. Such fish may be models for human diabetes and are therefore useful fish in the methods of the present invention. A drug screen can be performed using the methods described in the present invention by looking for chemicals test compounds that improve endocrine pancreas development in, for example, floating head mutants. In situ hybridization with endocrine pancreas markers, e.g. insulin, glucagon, somatostatin, islet-1, could be used as the method of detection.

Obesity: Overeating in teleosts, such as zebrafish, can be detected by feeding a meal of one color and then immediately feeding again with food of a different color. Liedtke, W., et al., Large-scale screening for alterations in zebrafish thermoregulation and food intake behavior in “Zebrafish Development and Genetics,” abstract book for the Apr. 26-30, 2000 Meeting at Cold Spring Harbor, N.Y. p. 168. The color can be observed through the transparent stomach of the zebrafish. Mutant fish with overeating behavior will continue to eat when wildtypes stop. The screen of the present invention would look for compounds that ameliorate overeating behavior.

In the methods of the present invention, a variety of test compounds from various sources can be screened for the ability of the compound to alter a phenotype associated with a disease or to test the effectiveness of a compound believed to be useful in treating a disease. Compounds to be screened can be naturally occurring or synthetic molecules. Compounds to be screened can also be obtained from natural sources, such as, marine microorganisms, algae, plants, and fungi. The test compounds can also be minerals or oligo agents. Alternatively, test compounds can be obtained from combinatorial libraries of agents, including peptides or small molecules, or from existing repertories of chemical compounds synthesized in industry, e.g., by the chemical, pharmaceutical, environmental, agricultural, marine, cosmetic, drug, and biotechnological industries. Test compounds can include, e.g., pharmaceuticals, therapeutics, agricultural or industrial agents, environmental pollutants, cosmetics, drugs, organic and inorganic compounds, lipids, glucocorticoids, antibiotics, peptides, proteins, sugars, carbohydrates, chimeric molecules, and combinations thereof.

Combinatorial libraries can be produced for many types of compounds that can be synthesized in a step-by-step fashion. Such compounds include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. In the method of the present invention, the preferred lest compound is a small molecule, nucleic acid and modified nucleic acids, peptide, peptidomimetic, protein, glycoprotein, carbohydrate, lipid, or glycolipid. Preferably, the nucleic acid is DNA or RNA.

Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources including, e.g., the DIVERSet E library (16,320 compounds) from ChemBridge Corporation (San Diego, Calif.), the National Cancer Instilute's (NCI) Natural Product Repository, Bethesda, Md., the NCI Open Synthetic Compound Collection; Bethesda, Md., NCI's Developmental Therapeutics Program, or the like.

In the methods of the present invention, a test compound to be screened for the ability of the compound to alter a phenotype associated with a disease or to test the effectiveness of a compound believed to be useful in treating a disease can be administered to the teleost by adding the test compound directly to the medium containing the live teleost. Alternatively, the test compound can first be dissolved in the medium and the live teleost submerged in the media subsequently. Such approaches have been used to introduce anesthetics and other chemicals to fish embryos. See, e.g., M. Westerfield, THE ZEBRAFISH BOOK: A GUIDE FOR THE LABORATORY USE OF ZEBRAFISH (3d. ed. 1995), which is incorporated herein in its entirety for all purposes. Test compounds can also be administered to the teleost by using microinjection techniques in which the agent is injected directly into the live teleost. For example, test compounds can be injected into either the yolk or body of a teleost embryo or both.

Test compounds can also be administered to teleosts by electroporation, lipofection, or ingestion or by using biolistic cell loading technology in which particles coated with the biological molecule are “biolistically” shot into the cell or tissue of interest using a high-pressure gun. Such techniques are well known to those of ordinary skill in the art. See, e.g., Sambrook et al., supra; Chow et al., Amer. J. Pathol. 2(6): 1667-1679 (1998).

Test compounds can be administered alone, in conjunction with a variety of solvents (e.g., dimethylsulfoxide or the like) or carriers (including, e.g., peptide, lipid or solvent carriers), or in conjunction with other compounds. Test compounds can be administered to the teleost before, at the same time as, or after administration of a dye used for detection of the response in the animal indicating a specific activity (e.g., cell death activity, angiogenesis activity, toxic activity).

A variety of techniques can be used to detect an alteration in the phenotype. Such techniques, include, for example, in situ hybridization, antibody staining of specific proteins (e.g., P-H3 staining), antibody markers that label signaling proteins. Alterations in phenotype can also be detected by, e.g., visual inspection, colorimetry, fluorescence microscopy, light microscopy, chemiluminescence, digital image analyzing, standard microplate reader techniques, fluorometry, including time-resolved fluorometry, visual inspection, CCD cameras, video cameras, photographic film, or the use of current instrumentation such as laser scanning devices, fluorometers, photodiodes, quantum counters, plate readers, epifluorescence microscopes, scanning microscopes, confocal microscopes, flow cytometers, capillary electrophoresis detectors, or by means for amplifying the signal such as a photomultiplier tube, etc. Responses can be discriminated and/or analyzed by using pattern recognition software. Compounds are identified and selected using the screening methods according to the activities and responses they produce.

Automated methods can be readily performed by using commercially available automated instrumentation and software and known automated observation and detection procedures. Multi-well formats are particularly attractive for high through-put and automated compound screening. Screening methods ian be performed, for example, using a standard microplate well format, with at least one zebrafish embryo in each well of the microplate. This format permits screening assays to be automated using standard microplate procedures and microplate readers to detect alteration of phenotype in the zebrafish embryos in the wells. A microplate reader includes any device that is able to read a signal, such as color, fluorescence, luminescence, radioactivity, or shape of the object from a microplate (e.g., 96-well plate). Methods of detection include fluorometry (standard or time-resolved), luminometry, or photometry in either endpoint or kinetic assays. Using such techniques, the effect of a specific agent on a large number of teleosts (e.g., teleost embryos) can be ascertained rapidly. In addition, with such an arrangement, a wide variety of compounds can be rapidly and efficiently screened for their respective effects on the cells of teleosts contained in the wells.

Sample handling and detection procedures can be automated using commercially available instrumentation and software systems for rapid reproducible application of dyes and agents, fluid changing, and automated screening of target compounds. To increase the throughput of a compound administration, currently available robotic systems can be used. Such systems include, e.g., the BioRobot 9600 from Qiagen Inc., Valencia, Calif.; the ZYMATE® from Zymark Corporation, Hopkinton, Mass.; and the BIOMEK® from Beckman Instruments, Inc., Fullerton, Calif. Most of the robotic systems use the multi-well culture plate format. Automated systems are useful in the processing procedures involving a large number of fluid changes that must be performed at defined time points.

In yet another aspect, the invention provides a compound obtained by the methods of screening and testing effectiveness of a test compound as outlined above. Useful compounds include, but are not limited to compounds described in the FIGS. 11A-11C. FIG. 11A shows that “8G16” (Group I) prevents the crash and burn cell cycle phenotype through 24 hours of development without affecting normal embryos. This compound, Adamantane-1-carboxylic acid (3-hydroxy-pyridin-2-yl)-amide, is a candidate agent for cancer chemotherapy and/or chemoprevention. FIG. 11B shows eight compounds (2-9), Group II including:
(2) 4-(4-Allyloxy-3,5-dibromo-benzenesulfonyl)-2,6-dibromo-phenol
(3) 4-Hydroxy-3-[3-(4-hydroxy-phenyl)-acryloyl]-6-methyl-pyran-2-one
(4) 2-Benzoyl-3a,7a-dihydro-indene-1,3-dione
(5) Toluene-4-sulfonic acid 2,4-dinitro-phenyl ester
(6) 3,5-Diiodo-N-[2-chloro-5-(4-chloro-benzenesul fonyl)-phenyl]-2-hydroxy-benzamide
(7) 1-(2-Amino-4-nitro-phenylamino)-3-phenyl-urea
(8) 1-(3,4-Dichloro-phenyl)-2-(2-imino-2H-pyridin-1-yl)-ethanone
(9) 2-(2-o-Tolyloxy-acetylamino)-benzoic acid, that decrease P-H3 staining in crash and burn embryos and also decrease staining in wildtype embryos. Therefore, they likely arrest the cell cycle in G1, S or early G2 when hisione H3 is not phosphorylated. This activity is analogous to the activity of aphidicolin in FIG. 4. These compounds are candidate agents for cancer chemotherapy. FIG. 11 C shows Group III compounds. These 6 compounds (10-15) increase P-H3 staining in wildtype embryos and include
(10) N-(2-Chloro-phenyl)-succinamic acid methyl ester
(11) 4-(2-Chloro-5-trifluoromethyl-phenylcarbamoyl)-butyric acid
(12) 4-(Naphthalen-1-ylamino)-3,5-dinitro-benzoic acid
((13) 2-[1-(3-Chloro-phenyl)-2,5-dioxo-pyrrolidin-3-ylsulfanyl]-N-(3-fluoro-phenyl)-acetamide
(14) 2-(5-Hydroxmethyl-8-metyl-3-oxa-bicyclo[3.3.1]non-7-en-2-yl)-phenol
(15) 5-Acetyl-4-(3-hydroxy-phenyl)-6-methyl-3,4-dihydro-1H-pyrimidin-2-one.
   Based on screens of the same library by other investigators at the ICCB, these results represent novel activities for these chemicals. These compounds are candidates for cancer chemotherapy.

It will also be appreciated by those skilled in the art that, although certain protected derivatives of compounds of formulas shown in FIGS. 11A-11C, which derivatives may be made prior to a final deprotection stage, may not possess pharmacological activity as such, they may be administered parenterally or orally and thereafter metabolized in the body to form compounds of the invention which are pharmacologically active. Such derivatives may therefore be described as “prodrugs”. All such prodrugs are included within the scope of the present invention.

The invention further encompasses compounds which are structurally similar to compounds shone in FIGS. 11A-11C, e.g., structural analogs, or derivatives thereof. Preferably, a derivative has at least 75%, 85%, 95%, 99% or 100% of the biological activity of the reference compound. In some cases, the biological activity of the derivative may exceed the level of activity of the reference compound. Derivatives may also possess characteristics or activities not possessed by the reference compound. For example, a derivative may have reduced toxicity, prolonged clinical half-life, or improved ability to cross the blood-brain barrier.

The invention also includes a method of treating a host having a cell cycle defect, e.g., cancer, comprising administering a compound obtained using the present invention or compounds 1-15 as set forth in FIGS. 11A-11C.

The methods disclosed herein provide for the parenteral or oral administration of a compound to a subject, such as a human, in need of treatment. Parenteral administration includes, but is not limited to, intravenous (IV), intramuscular (IM), subcutaneous (SC), intraperitoneal (IP), intranasal, and inhalant routes. In the method of the present invention, the compound is preferably administered orally. IV, IM, SC, and IP administration may be by bolus or infusion, and may also be by slow release implantable device, including, but not limited to pumps, slow release formulations, and mechanical devices. The formulation, route and method of administration, and dosage will depend on the disorder to be treated and the medical history of the patient. For parenteral or oral administration, compositions of the compound may be semi-solid or liquid preparations, such as liquids, suspensions, and the like.

The invention further provides a pharmaceutical composition comprising a compound obtained using the present invention or as set forth in FIGS. 11A-11C. Preferred compositions comprise, in addition to the compound, a pharmaceutically acceptable carrier. (i.e., sterile and non-toxic) liquid, semisolid, or solid diluent that serves as a pharmaceutical vehicle, excipient, or medium. Any diluent known in the art may be used. Exemplary diluents include, but are not limited to, water, saline solutions, polyoxyethylene sorbitan monolaurate, magnesium stearate, methyl- and propylhydroxybenzoate, talc, alginates, starches, lactose, sucrose, dextrose, sorbitol, mannitol, glycerol, calcium phosphate, mineral oil, and cocoa butter. Suitable carriers or diluents are described, for example, in the Remington: The Science and Practice of Pharmacy, by Alfonso R. Gennaro, ed. A. L. Gennaro, Lippincott, Williams & Wilkins; ISBN: 0683306472; 20th edition, Dec. 15, 2000, a standard reference text in this field, which is incorporated herein by reference in its entirety. Preferred examples of such carriers or diluents include, but are not limited to, water, saline, Ringer's solution, dextrose solution, and 5% human serum albumin. Liposomes and nonaqueous vehicles such as fixed oils may also be used. The formulations are sterilized by commonly used techniques.

The compositions, or pharmaceutical compositions, comprising the nucleic acid molecules, vectors, polypeptides, antibodies and compounds identified by the screening methods described herein, can be prepared for any route of administration including, but not limited to, oral, intravenous, cutaneous, subcutaneous, nasal, intramuscular or intraperitoneal. The nature of the carrier or other ingredients will depend on the specific route of administration and particular embodiment of the invention to be administered. Examples of techniques and protocols that are useful in this context are, inter alia, found in Id.

The dosage of these compounds will depend on the disease state or condition to be treated and other clinical factors such as weight and condition of the human or animal and the route of administration of the compound. For treating human or animals, between approximately 0.25 μg/kg of body weight to 100 mg/kg of body weight of the compound can be administered. Therapy is typically administered at lower dosages and is continued until the desired therapeutic outcome is observed.

The invention also provides an article of manufacture comprising packaging material and a pharmaceutical composition contained within said packaging material, wherein said packaging material comprises a label which indicates said pharmaceutical may be administered, for a sufficient term at an effective dose, for treating and/or preventing cancer, hematologic disease, immunologic disease, angiogenesis defect, bone disease, cardiovascular disease, obesity, diabetes, or neurodegenerative disease in a mammal, wherein the pharmaceutical composition comprises a compound obtained using the present invention or as set forth in FIGS. 11A-11C.

The present invention is further illustrated by the following examples, which should in no way be construed as being further limiting. The contents of all references, pending patent applications and published patent applications, cited throughout this specification including the examples are hereby incorporated by reference in their entirety.

### REFERENCE EXAMPLES

Fish mutations discussed in the specification as well as mutants, which represent new model diseases can be created using the methods outlined as follows.

ENU mutagenesis: Adult male zebrafish of the wik-background were mutagenized with ENU and mated to wild-type females of the same background. The ENU mutagenesis was performed essentially as described in van Eeden et al. [Methods Cell Biol 60: 21-41, 1999]. Shortly, male zebrafish are exposed to about 2.5-3.0 mM ENU in Embryo medium for one hour at 25° C. Fish are washed to two changes of fish aquarium water for one hour each wash. The treatment can be repeated about 3 and 6 days later. After exposure to mutagens, male fish are mated weekly to wild-type female fish. The F1 progeny generated 4-24 weeks after the last ENU treatment are used for screening.

Creation of haploid embryos: The F1 helerozygote females harboring point mutations created using ENU mutagenesis described above were squeezed to produce haploid eggs that were fertilized with UV inactivated sperm, yielding haploid embryos.

The F1 female fish were placed in isolation chambers with a male fish overnight. The next morning, prior to egg laying, the males were removed. The females were individually anesthetized with 0.02% Tricahe, and their eggs were removed by gentle pressure on the abdomen. The eggs were mixed with 2.0 microfilters of VU-inactivated sperm. After one minute embryo water was added. The embryos were subsequently incubated at 28.5° C.

Whole mount immunohisiochenmical staining of zebrafish embryos: The haploid embryos were screened at 36 hours with an anti-phospho histone H3 antibody to screen for potential cell cycle mutants. Clutches were analyzed under a stereo dissecting microscope and scored for an abnormal number of stained cells in 50% of the embryos. The parental F1 females from those clutches with 50% abnormally staining embryos were set aside.

750 F1 female zebrafish were screened resulting in identification of 41 mutant clutches: 21 had increased staining, 11 had decreased staining and 9 had other phenotypes, such as focal staining.

There are several alternative fixation methods that can be used before staining. Here, the embryos were fixed 4 hours in 4% paraformaldehyde. After fixation, the embryos were stained with an antibody recognizing the phosphorylated histone H3 (pH3).

The staining was performed using a peroxidase method. The embryos were fixed and stored in 5 ml glass vials. The embryos were first dechlorinate using watchmaker forceps or pronase treatment. Pronase treatment is faster for large batches of embryos. To dechlorinate the embryos using pronase, 2 mg of pronase was added on them in E3 medium.

The preparation was swirled at room temperature until about 80% of the chorions were removed after which the preparation was rinsed 3-4 times with E3.

Embryos were fixed with 4% paraformaldehyde/PBS overnight at 4° C. and consequently washed twice in PBS.

Staining with antibody was performed by first incubating the fixed embryos for 7 minutes in −20° C. acetone in glass vials. The embryos were rinsed once in double distilled water and twice in PBS for one minute in each after which they were washed 2 times 5 minutes in PBS with 0.1% Tween-20 (PBST).

Unspecific binding was blocked by incubating embryos for 30 minutes to one hour at room temperature with PBST and blocking reagents (10% heat treated lamb serum, 2% blocking reagent diluted from a 10% stock (Boehringer-Mannheim Biochemicals (Roche)) and 1% DMSO.

Primary anti phospho histone H3 antibody was diluted to 1 ug/ml in PBST/black reagents/DMSO and incubated overnight at 4° C. or at room temperature for 2-4 hours. Primary antibody was removed and the preparation washed 4 times 15 minutes in PBST. Secondary anti-rabbit IgG antibody conjugated to horse radish peroxidase (HRP; Jackson Immunoresearch) at 1:300 in PBST/block reagents/DMSO was added to the embryo preparation and incubated overnight at 4° C. or room temperature for 4 hr.

Detection of staining was performed after rinsing once and then washing for 30 minutes with PBST and 10% heat treated lamb serum and three times 30 minutes in PBST. The DAB stain was added at appropriate dilution and stained for 10 minutes to overnight wrapped in foil to protect from light. Often a staining time of 1 to 5 minutes was adequate. After staining the preparation was washed two times 5 minutes in PBST and fixed in 4% paraformaldehyde/PBS overnight at 4° C. The stained preparations were stored in fixative at 4° C. or alternatively in methanol. The preparations were mounted in 90% glycerol, 10% 1×PBS and photographed. Alternatively, the preparation can be dehydrated and mounted. Dehydration can be performed with washing with 100% MetOH twice, 10 minutes each, followed by a 2:1 mixture of benzylbenzoate:benzylalcohol wash. This mixture has the same refractive index as yolk, and clears the embryos well but it is not viscous like glycerol and embryos are hard to position.

Histone H3 phosphorylation has long been implicated in chromosome condensation during mitosis (Strahl, B. D., et al., Nature, 403: 41-45, 2000). Phosphorylation at Ser 10 of hisione H3 is tightly correlated with chromosome condensation during both mitosis and meiosis (Mendzel et al. Chromosome 106: 348-360, 1997). Phosphorylation at this site is also required for the initiation of the chromosome condensed state, as well as the induction of immediate-early genes such as c-jun, c-fos and c-myc (Strahl, B. D., et al., Nature, 403: 41-45, 2000; Spencer, V. A., et al., Gene, 240: 1-12, 1999). PKA, Rsk-2 and MSKI are required for H3 phosphorylation (Id.). Phospho-Histone (Ser10) Antibody detects Histone H3 when it is phosphorylated at serine 10. It is a useful tool to identify the phosphorylation of H3 and monitor cell mitosis and meiosis by immunocytochemistry.

The pH3 antibody stains cells known to be proliferating in zebrafish embryos. Stained cells were distributed throughout the embryo at 12 and 16 hours post fertilization (hpf) and increased in number from 24-48 hpf. As each organ undergoes proliferation during distinct developmental stages, pH3 staining increases. There was a particularly high concentration of staining in the eye and developing nervous system 24-48 hpf. High magnification views of these stained embryos showed many mitotic figures demonstrate that pH3 antibody stains cells undergoing mitosis. The stained cells in the eye were different from cells in the lens that undergo apoptosis. Staining of later stage embryos has proven unsuccessful, although it is unclear whether this is a result of a decrease in pH3 levels or a decrease in the permeability of the embryo to the pH3 antibody.

Staining performed on haploid embryos also delineated mitotic cells. To demonstrate the specificity of pH3 antibody for cycling cells, we tested pH3 staining in embryos that were irradiated. Irradiation induces a checkpoint after which cells subsequently begin to cycle. After irradiation, pH3 staining decreased to a nadir at 30 minutes, and recovers to near normal levels by 2 hours.

Whole mount in situ analysis of zebrafish embryos: The whole mount in situ analysis was performed essentially as described by S. Schulte-Merker, J. H. Odenthal, and C. Nüsslein-Volhard (The Zebrafish Science Monitor. 2, Sep. 21, 1992 at zfish.uoregon.edulzf_info/monitor/vol2.1vol2.1.html).

The embryos were dechorionated using watchmaker forceps or pronase treatment and fixed with 4% paraformaldehyde/PBS overnight at 4° C. as described above. The dechorionated embryos were washed 2 times in PBS for 5 minutes at room temperature. The washed embryos were transferred to vials with 100% methanol and incubated for 5 minutes. Methanol was replaced with fresh 100% methanol and put at −20° C. for at least 20 minutes.

The dechorionated embryos were rehydrated and fixed at room temperature. Embryos were processed in batches according to age (proteinase K treatment) and later separated. Either 5 ml vials or 12 well plates. Each wash was 2 to 3 ml in the vials or 50 ml in the well trays: 5 minutes in 50% MetOH in PBST, 5 minutes in 30% MetOH in PBST and 2 times in PBST, 5 minutes each (dechorionating embryos can also be done at this point, but chorions are sticky after having been in MeOH). The rehydrated embryos were fixed for 20 minutes in 4% paraformaldehyde in PBS and washed with 2 times PBST (PBS, 0.1% Tween) for 5 minutes each.

The dechorionated preparations were digested with proteinase K (10 μg/ml in PBST) at room temperature for about 5 minutes (time can vary from 1 minute up to 10 hours), 10 minutes (10-24 hours) or 15 minutes (20 μg/ml in PBST)(>24 hours). After digestion, the preparations were rinsed briefly in PBST; washed once in PBST for 5 minutes and fixed as described above; and washed again two times in PBST as described above.

Up to 200 embryos were transferred into 1.5 ml microfuge tubes in PBST. PBST was removed so that the embryos are just covered and add approximately 500 μg HYB⁻ solution (50% formamide, 5×SSC, 0.1% Tween-20). Hybridization steps were performed in a water bath or preferably in a hybridization oven without rocking. The preparation was allowed to incubate 5 minutes at 60° C whereafier HYB⁻ was replaced by an equal volume of HYB+ (HYB⁻, 5 mg/ml torula (yeast) RNA, 50 μg/ml heparin). Prehybridization was performed at 60° C. for 4 hours in HYB+ (overnight prehybridization was sometimes preferred). About 5 to 10 μg of a linearized plasmid was used and probes shorter than 2500 nucleotides were not hydrolyzed.

Hybridization was performed by adding 100 ng RNA probe to 500 μl fresh HYB+ and heated for 5 minutes at 68° C. The probe in HYB+ was added and the preparation was incubated overnight or about 12 hours at 60° C. whereafier the probe was removed.

The following GATA-2 and TTG2 steps were performed on 24 well plates using prewarmed solutions.

GATA-2 probe was the most common starting point. The following incubations were performed: 2×30 minutes at 60° C. in 50% formamide/2×SSCT (SSC, 0.1% Tween); 1×15 minutes at 60° C. in 2×SSCT; and 2×30 minutes at 60° C. in 0.2×SSCT.

TTG2 probe was used to decrease background. The following incubations were performed: 30 minutes at 60° C. in 50% formamide/50% 2×SSCT; 3×10 minutes at 37° C. in 2×SSCT; 1×5 minutes at 37° C. in PBST; 30 minutes at 37° C. in RNAse A, 20 μg/ml, RNAse T1, 100 U/ml in PBST solution; 10 minutes at 37° C. in 2×SSCT; 60 minutes at 60° C. 50% formamide/50% 2×SSCT; 15 minutes at 60° C. 2×SSCT; and 2×15 minutes at 50° C. in 0.2×SSCT.

The detection of staining was performed as follows. The embryo preparation was washed 2×5 minutes in MABT (100 mM maleic acid, Sigma M0375, St Louis, Mo.; 150 mM NaCl, 55 g TRIS for 2 L final, pH 7.5 combined with 0.2% Tween-20). The preparation was blocked for one hour at room temperature with MABT plus blocking reagents (10% heat treated lamb serum, 2% BMB 1096 176, Boehringer-Mannheim Biochemicals, Indianapolis, Ind.; blocking reagent in 100 mM maleic acid, Sigma M0375; 150 mM NaCl, 55 g TRIS for 2 L final, pH 7.5. Fab-AP as (Boehringer-Manaheim Biochemicals) was added at a 5000-fold dilution and shaken overnight at 4° C. in MABT plus blocking reagents.

The preparation was rinsed once then wash 30 minutes with MABT and 10% heat treated lamb serum and once again with 5×30 minutes in MABT. Embryos were washed 3×5 minutes in staining buffer 100 mM Tris, pH 9.5, 50 mM MgCl₂, 100 mM NaCl, 0.1% Tween-20, 1 mM Levamisole. Embryos were stained at room temperature in BMB purple (Boehringer-Mannheim Biochemicals) and 5 mM fresh levamisole hydrochloride for 30 minutes to overnight. Embryos were washed two times for 5 minutes in PBST and fixed overnight and stored in 4% paraformaldehyde/PBST at 4° C. For photography, the embryos were placed in 70% glycerol 30% 1×PBST.

Flow cylometric cell sorting analysis of zebrafish embryos to identify defects in cell cycle: To analyze the DNA content of the embryos wild-type and mutant embryonic cells were subjected to DNA flow cytometric cell sorting (FACS). We have shown that the FACS analysis of DNA content can be performed on cells from a single embryo allowing analysis and comparison of mutant and wild-type cell cycle phenotypes.

Embryos were anesthetized with tricaine (3-amino benzoic acid ethylester also called ethyl m-aminobenzoate, in a powdered form from Sigma, Cat.# A-5040). Tricaine solution for anesthetizing fish was prepared by combining the following: 400 mg tricaine powder, 97.9 ml DD water, and about 2.1 ml 1 M Tris (pH 9), pH was adjusted to about 7. Before use 4.2 ml of Tricaine solution was mixed with 100 ml clean tank water.

The embryos were dechorionated as described above and resuspended in a small volume of DMEM −20% FBS in a microtube. Embryos were disaggregated and resuspend in 1-2 ml of DMEM+20% FBS. The solution was passed through 105 μm mesh, and consequently 40 μm mesh. The total volume was raised to 5 ml and the cells' in the sample was counted using hemocytometer. Volume equaling 2×10⁶ cells was transferred in 15 ml conical tube and filled to a total volume of 5 ml with PBS. The sample was spinned at 1200 rpm for 10 minutes and the liquid was aspirated off. 2 ml P1 solution (0.1% Sodium Citrate, 0.05 mg/ml propidium iodide, 0.0002% Triton X100 and 2 μg of RNase) was added. The sample was incubated in dark at room temperature for 30 minutes before transferring on ice and sorting on a FACS analyzer.

Gamma radiation induced a cell cycle arrest in zebrafish embryos as seen by DNA content analysis by FACS. Cell cycle arrest in early G2 produced both the increase in cells with 4N DNA content and the decrease in the number of mitotic cells. Flow cytometric analysis of 24 hours post fertilization zebrafish embryos demonstrated accumulation of cells in G2-phase, indicating activation of the G2 DNA-damage checkpoint. Consistent with the known kinetics of eukaryotic DNA repair, reversal of G2 arrest was seen beginning at 2 hrs post-radiation. During this same time period, pH3 immunoreactivity was profoundly depressed, suggesting that the G2 radiation checkpoint preceded the onset of chromatin condensation and H3 phosphorylation.

The analysis of SQW 226 (the crash&burn mutant fish), and SQW 280 demonstrated endoreduplication, a feature commonly found in human tumors such as neuroblastoma, suggesting that the increased pH3 staining in whole mount truly indicated an increase of cells at the G2/M boundary in vivo. The DNA content analysis of mutants SQW 226, SQW 319 (the standstill mutant fish), and SQW 61 demonstrated aberrant cell cycles including the following characteristics: endoreduplication (SQW 226), populations of larger cells (SQW 226 and SQW 61), an increase in the G2/M population (SQW 319), and an increasein the G1 population (SQW 61). Decrease of G2 and increase in G1 population in SQW61 analysis suggested that the cells were arrested in G1 stage.

Analysis of apopiosis markers in zebrafish embryos to identify defects in apoptosis: Embryos were stained for 1 hr in acridine orange, washed in PBS and observed with fluorescein filter.

Apoptosis in zebrafish embryos can be detected using a variety of techniques. For example, acridine orange staining of SQW 226 demonstrated that the mutant has a significant increase in cell death at 24 or 36 hrs. Cells with defective cell cycle undergo an apoptotic death. Mutant SQW 226 demonstrated an increased number of cell undergoing cell death as compared with the wild-type. Heterozygous in-crosses of SQW 226 were performed. At 24 hours, it was apparent that one quarter of the clutch displays a “tail up” phenotype. These homozygous embryos were then stained with the vital dye acridine orange and examined under an epifluorescent microscope to evaluate the extent of apoptosis.

Lysotracker (Molecular Probes, Eugine, Oreg.) is an aldehyde fixable red dye that also stains apopiotic cells in live embryos, and allowed us to further study the mutants in conjunction with other probes. A significantly increased apoptosis in various zebrafish embryo mutants using Acridine Orange staining was shown.

BrdU staining of zebrafish embryos to identify defects in S phase: BrdU is incorporated into DNA by cells in S phase. The BrdU assay allowed further refinement of the cell cycle phenotype. Live 24 hours post fertilization embryos were incubated in 10 mM BrdU on ice, rinsed and chased for 0, 10, 30 and 60 minutes at 28.5° C. Details of labeling in the eye and tail demonstrated a progressive increase in labeled cells with longer incubations.

Both SQW 226 and 319 zebrafish mutants demonstrated decreased incorporation of BrdU. BrdU incorporation in wild-type and mutant embryos after a 10-minute chase period showed that S-phase cells are moderately decreased in SQW226 and severely decreased SQW 319. Summary of analysis of zebrafish mutants using pH3 staining, apoptosis markers, BrdU incorporation and FACS is shown in the following Table 1.

TABLE 1
Characterization of SQW mutants.

| | | | BrdU | |
|---|---|---|---|---|
| SQW Mutant | H3 staining | Apoptosis | incorp. | DNA flow |
| 61 | ↓ posteriorly | n.d. | ↓ | Increased cells |
| | | | | in G1 |
| 213 | ↑ neural/ | ↑ | n.d. | Normal |
| | pronephric duct | | | |
| 226 | ↑↑↑ | ↑↑ | ↓ | Polyploid |
| crash & burn | | | | |
| 280 | Large spots | n.d. | n.d. | Polyploid |
| 319 | ↓↓↓ | ↑ | ↓↓ | Increased cells |
| standstill | | | | in G2 |
| 332 | ↓↓ | n.d. | ↓↓ | n.d. |
| 333 | ↑ | n.d. | n.d. | n.d. |

n.d. = not determined.;
↑ = increased number of cell staining;
↓ = decreased staining.

Tubulin staining of zebtafish embryos to identify defects in mitosis: The mitotic spindle plays a vital role in cell cycle, and the mutants could represent defects in this process. Tubulin staining of the zebrafish for examining mitosis was performed. Disrupted zebrafish embryos were incubated on polylysine coated slides and air dried. The slides were incubated in PBST/Block (as described above) followed by incubation in fluorescein conjugated monoclonal anti-α-tubulin (Sigma) diluted 1:100 and washed in PBST. The slides were observed under microscope with a fluorescein filter. Defective spindle formation was shown in two mutants, SQW 280 and SQW 226.

Irradiation analysis of zebrafish embryos to identify checkpoint defective mutant: Zebrafish embryos were γ-irradiated 24-36 hours post fertilization with 800-1600 rads which causes a cell cycle arrest, yet the embryo recovers and continues to develop normally at least about to 24 hours of age. pH3 staining decreases substantially to being barely detectable by 30 minutes post radiation, but pH3 recovers to normal levels at 2 hours post radiation. DNA flow cytometric analysis demonstrates an increasing proportion of cells in G2/M from 15 minutes post radiation to 4 hours post radiation, suggesting a G2 arrest.

Eggs from 100 F1 females harboring mutations were squeezed and exposed to inactive sperm to create haploid embryos. The embryos were evaluated at 12 hours and irradiated at 14 hours with 1600 rads. One hour later the embryos were fixed as described above and stained for pH3. One mutant, R176 showed 50% mutant embryos with persistent pH3 staining suggesting a damaged radiation checkpoint.

We irradiated SQW 226 to evaluate whether SQW 226 mutant zebrafish strain has checkpoint defects. SQW 226 mutant zebrafish did not show a decrease in the number of mitotic cells as the homozygous mutants fail to display decreased pH3 staining. Therefore, either SQW226 is able to override a checkpoint or alternatively exhibits an exit block which suggests that either SQW 226 is resistant to the radiation-induced cell cycle arrest or the cell cycle is blocked and shows no effect from radiation. In contrast, wild-type embryos (+/−or +/+) had decreased pH3 staining after irradiation. Each mutant was evaluated in this irradiation screen for cell cycle checkpoint defects.

In addition, this irradiation screen forms the basis for doing a checkpoint or exit block screen on zebrafish embryos. A haploid screen that was performed based on the observed radiation-induced cell cycle arrest. Haploid embryos from F1 females, which is the progeny of ENU treated males and wild-type females, was irradiated and fixed 45 minutes post radiation. These embryos were stained with the pH3 antibody and mutants that did not exhibit the normal decrease in mitotic cells can be identified. These mutants are likely to affect cell cycle machinery or checkpoint control genes and are excellent models for the study of cancer formation and as subjects for future modifier screens.

Creation and analysis of diploid embryos: The 41 F1 wik-ENU female zebrafish representing the potential mutations were outcrossed to wik males. The resulting F2 progeny was raised to adulthood and in-crossed to re-identify heterozygote pairs and to confirm that the pH3 phenotype can be recapitulated in the diploid state.

We identified the progeny from 29 F1 females that have been in-crossed (20 matings each). In this analysis, heterozygote pairs for seven mutations (SQW 61, 213, 226, 280, 319, 332, 333) were identified. The SQW 226 mutant had increased pH3 staining. Counting cells in the body and tail (n=5) demonstrated 2.2 fold more stained cells in the mutant compared to wild-type. The diploid phenotypes for these mutants resembled the haploid phenotypes. SQW 213 also had increased staining but in a focal distribution in neural cells and in the pronephric duct. SQW 319 has decreased pH3 staining, and SQW 61 had only slightly increased staining; SQW 280 had a larger domain of nuclear staining with fewer cells staining. Map crosses for all 41 F1 females (wik.ENU heterozygous female crossed to a wild-type AB male) were also generated.

Given average mutant recovery rates from haploid screens that we performed, the pilot screen will recover at least 15-20 mutants affecting the cell cycle. In some mutants, there was an increase in pH3 staining diffusely. In these mutants, there was a decrease in the size of the head and a curved up tail. Other mutants had decreased pH3 staining and appeared smaller than control siblings.

Positional cloning of genes involved in cell cycle regulation. The mutants were mapped onto zebrafish linkage groups by either determining centromeric linkage by half-tetrad analysis (Johnson, S. L., et al. Genetics, 139: 1727-1735, 1995) or by scanning microsatellites for linkage. This half tetrad method involved following the segregation of known SSLP centromeric markers with respect to wild-type and mutant gynogenetic diploid embryos (Streisinger, G., et al., Nature, 291: 293-296, 1981; Streisinger G., et al., Genetics, 112: 311-319, 1986).

The mutation can also be assigned to a linkage group, by bulk segregation analysis with CA repeat markers (Talbot W. et al. in Methods in Cell Biology eds. H. I. Detrich, M. Westerfield, L. Zon, Academic Press, San Diego: 260-284, 1999; Liao, E. et al. Id. at 181-183). A wik background fish carrying the mutation (heterozygote) is mated to a polymorphic strain (AB). Haploid embryos are generated from heterozygous wik/AB hybrid females by fertilizing eggs with UV-irradiated sperm. Alternatively, diploid embryos can be generated by mating heterozygous hybrid males and females. Either haploid or diploid embryos are scored as either wild-type or mutant by fixing and staining them with the anti-pH3 antibody. DNA is then made from individual embryos. Bulk segregation analysis is performed on wild-type and mutant pools of 20 DNA samples (two wild-type pools and two mutant pools). PCR will then be performed on these pools using CA repeat primers from the linkage group indicated. Bands that amplify from both AB and wik DNA are uninformative; however, bands that are polymorphic between the two strains can be used as positional markers. A linked marker will be identified as one that segregates in the pools, meaning that bands of different sizes are amplified from the wild-type as compared to the mutant pool. If a linked marker is found, it will be tested on individual embryos to determine the recombination frequency between the marker and the mutation.

Using this approach, we genotyped 600 mutant embryos and mapped SQW226(crash&burn) to chromosome 11 of the Zebrafish. A marker within 1.2 cM of the mutation was isolated (8/612 embryos). Because there are only 3000 CA markers currently available it may be necessary to screen other markers because a closely flanking marker may not be found. AFLP analysis has proved to be a useful way to test many markers simultaneously. Testing 256 primer combinations can yield information on 6400 loci (Ghebranious N., et al., Oncogene, 173385-3400, 1990).

Using linkage analysis, the following six mutants were located in zebrafish genome map: SQW 61 was mapped on chromosome 2; SQW 213 was mapped on chromosome 8; SQW 226 was mapped to chromosome 11; SQW 280 was mapped to chromosome 6; SQW 319 (standstill) was mapped to chromosome 13; and SQW 333 was mapped to chromosome 15. Mutants SQW 61 and SQW 213 are flanked with markers that can be analyzed on an agarose gel.

1664 mutant embryos for SQW226 mutant zebrafish strain were collected and the ESTs in the critical interval were tested for recombination using linkage analysis. Six recombinants were obtained out of the 1664 mutant embryo DNAs that were tested. The recombinant fish are used for a chromosomal walk to identify the SQW 226 gene. (Talbot and Schier, Methods Cell Biol 60: 260-287, 1999).

Cloning of unknown genes is performed from libraries including BACs, PCAs, or YACs as described, for example in Amemiya et al. (Methods Cell Biol 60: 236-259, 1999). Mutation detection, nucleic acid sequencing and sequence analysis can be performed using techniques well known in the art and described in detain in for example Molecular Cloning: A Laboratory Manual. Third Edition by Joe Sambrook, Peter MacCallum, David Russell, CSHL Press, 2001.

Carcinogenesis assay: Carcinogenesis assay is used to determine which mutants are relevant to development of tumors or cancer. The assay will show whether zebrafish mutants that have abnormal cell cycle according to the haploid embryo screening described above are more prone to developing cancer than their wild-type siblings. The carcinogen should accelerate tumor development in these fish.

Both mutant and wild-type 3-week-old fish are exposed to the carcinogens 7, 12 Dimethyl benzanlhracene (DMBA) at doses of about 1.0, 2.0, 5 and 10 ppm and N-methyl-N-nitro-N-nitrosoouanidine (MNNG) at doses of about 0.5, 1.0, 2.0 and 3.0 ppm for an approximately 24-hour period and then placed into fresh water and raised to adulthood. Survival of the fish is monitored and fish that die or look ill are fixed for sectioning. Alternatively, an entire cohort can be fixed for sectioning and histologic analysis of tissues at an arbitrary time point which is usually about 7 months.

Carcinogen-treated zebrafish develop, for example, medulloblastoma or germ cell tumors that closely resembles human disease as shown in FIG. 4. Wild-type fish were with DMBA and MNNG. 9/86 or 10.4% fish treated with DMBA developed tumors and 10/128 or 7.8% of the fish treated with MNNG developed tumors. DMBA resulted in more brain and liver tumors whereas MNNG yielded more mesenchymal and testicular tumors. Mung: 0.5, 1.0 and 2.0 ppm; DMBA: 2.5, 5.0 and 10.0 ppm.

To evaluate rates of spontaneous and carcinogen induced tumorigenesis in mutant strains, the 21 day-old fry from incrosses were exposed for 24 hours to either vehicle control (DMSO) or 5.0 ppm DMBA. The early death rate observed in the mutants resulted in analyzing the fish at 3 months rather than 6 months which was originally estimated as appropriate. Several of the mutants show an increase in tumor incidence compared to the wild-type as can be seen in the Table 2 below.

TABLE 2
Summary of the results form the carcinogenesis assay.

| | DMSO | | | DMBA | | |
|---|---|---|---|---|---|---|
| Genotype | #tumors | #treated | % | tumors | treated | % |
| WT* | 0 | 35 | 0 | 2 | 9 | 5 |
| SQW 61 | 0 | 6 | 0 | 4 | 132 | 8 |
| SQW 213 | 1 | 64 | 2 | 2 | 28 | 7 |
| SQW 226 | 0 | 61 | 0 | 4 | 20 | 20 |
| crash&burn | | | | | | |
| SQW 280 | 1 | 43 | 2 | 6 | 47 | 12 |
| SQW319 | 1 | 10 | 10 | n.d. | — | — |
| standstill | | | | | | |
| SQW 333 | 2 | 31 | 6 | n.d. | — | — |

n.d. = not determined;
*Wild-type data are from 6 months post-treatment. The mutant strains were analyzed three months post-treatment.

Tissue sections from a medulloblastoma in a fish treated with (7,12) dimethylbenzanthracene were compared to wild-type using low power view under a light microscope. Low resolution indicates 40×, medium 200× and high 400× magnification. A medium and high resolution views show the similarity of fish and human tumors. For example, a genn-cell tumor in a fish treated with N-methyl-N′-nitrosoguanidine closely resembled the liver and testis tumors, respectively.

### EXAMPLES

The Zebrafish Cell Cycle: The basic molecular machinery of the cell cycle is well conserved through evolution—so much so that yeast have been a good model for the mammalian cell cycle. Some of the cell cycle machinery in zebrafish has been shown to be homologous to mammalian systems. For example, cyclin D1 has been cloned in zebrafish and its amino acid sequence is 77% identical to the human homologue. Yarden, A., D. Salomon, and B. Geiger, Zebrafish cyclin D1 is differentially expressed during early embryogenesis. Biochim. Biophys. Acta 1264, 257-60 (1995). Within the cyclin box region (a feature of G1 cyclins), the homology is even more striking—88% identitical. There are also numerous expressed sequence tags (EST's) of cell cycle genes present in the zebrafish database at Washington University, St. Louis, Mo.

The zebrafish embryonic cell cycle exhibits similarities to the Xenopus and Drosophila cell cycle. Zebrafish embryos begin dividing synchronously and rapidly (approximately 15 min cell cycles) until they reach mid-blasiula transition (MBT) which occurs after about 10 cell divisions. Kane, D. A., Cell cycles and development in the embryonic zebrafish. Methods Cell Biol. 59, 11-26 (1999). At that point, zygotic transcription begins and the cell cycle becomes asynchronous and slower. Three mitotic domains are established, each with different average cell cycle times. Kane, D. A., R. M. Warga, and C. B. Kimmel, Mitotic domains in the early embryo of the zebrafish. Nature 360, 735-737 (1992). Also notable at MBT is the onset of characteristic checkpoint type responses and the capacity to undergo apoptosis in response to cell cycle perturbing chemicals. For example, treating post-MBT embryos with nocodazole causes metaphase arrest and apoptosis. Ikegami, R., J. Zhang, A. K. Rivera-Bennetts, and T. D. Yager, Activation of the metaphase checkpoint and an apoptosis programme in the early zebrafish embryo, by treatment with the spindle-destabilising agent nocodazole. Zygote 5, 329-350 (1997). Metaphase arrested cells can be driven into G1 by adding the calcium-specific ionophore A23187. Several chemicals that cause S-phase arrest and apoptosis in mammalian cells, such as camptothecin, hydroxyurea, and aphidicolin, have been shown to cause apoplosis in zebrafish embryos. Ikegarni, R., P. Hunter, and T. D. Yager, Developmental activation of the capability to undergo checkpoint induced apoptosis in the early zebrafish embryo. Dev. Biol. 209, 409-433 (1999).

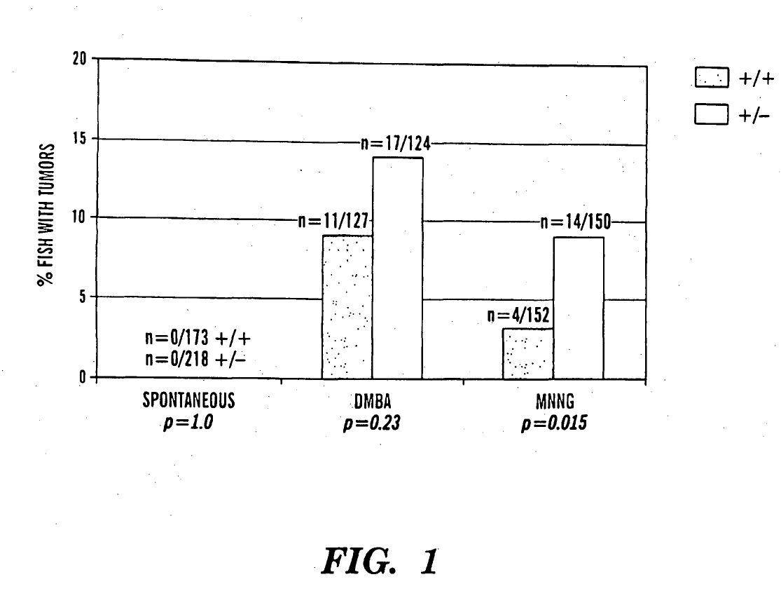

We have identified eight zebrafish cell cycle mutants, which were created using the methods described in the Reference Examples above. The cell cycle defects are observed in homozygous mutant embryos which die by day 5 of development. Heterozygotes generally appear unaffected, but ongoing carcinogenesis assays are showing that some cell cycle mutants have an increased incidence of cancer. Crash & burn heterozygotes (SQW 226) have a statistically significant increase in cancer both spontaneously and in the presence of carcinogens (FIG. 1). Given that crash & burn can therefore be considered a cancer model, we focused on screening for chemicals that can revert the cell cycle defect in crash & burn homozygous mutant embryos. Chemicals that revert or improve the cell cycle defect will be tested on heterozygotes for chemopreventive or chemotherapeutic activity. Some screening was also done on the mutant standstill (SQW 319), which has an interesting cell cycle defect. The carcinogenesis data with standstill heterozygotes suggest an increased susceptibility to cancer, but the data are not statistically significant at this time.

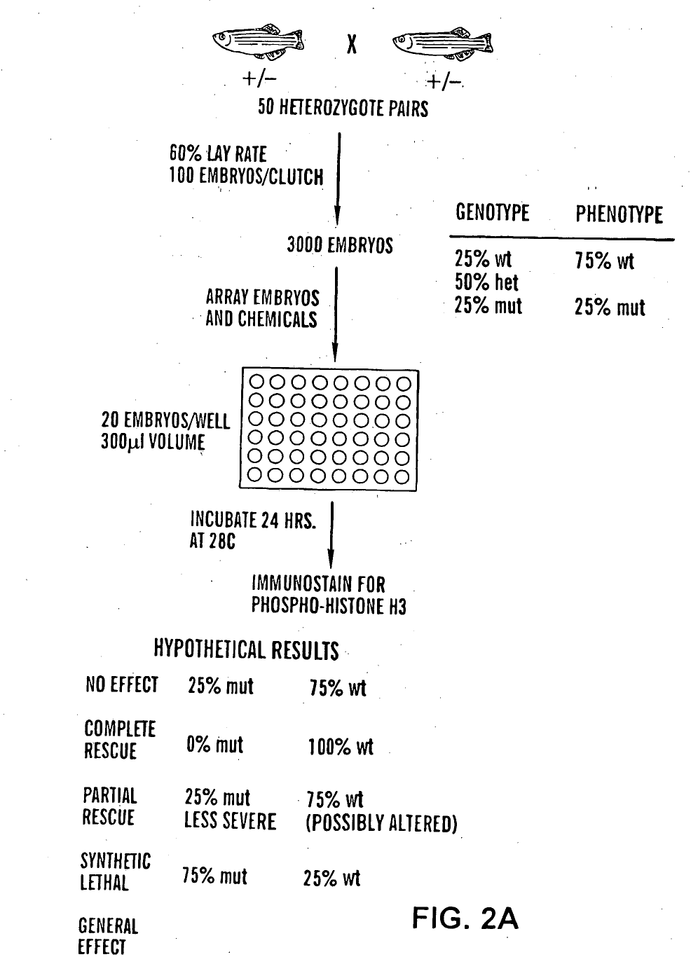

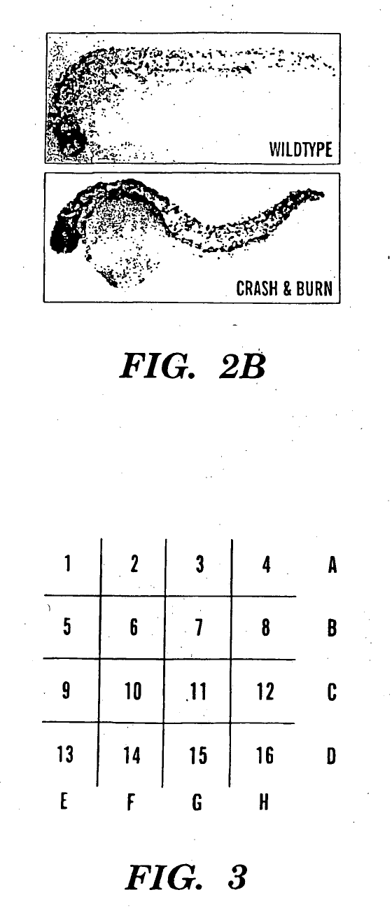

Fish: Given that all of the mutants are lethal by embryonic day 5, homozygous mutant embryos were generated by incrossing adult heterozygotes. About 50 heterozygote pairs of crash & burn (or in some cases standstill) were mated weekly, generating about 3000 embryos per week (FIG. 2). These embryos were composed of a Mendelian distribution of 25% homozygous mutants, 50% heterozygotes and 25% wild-types. The clutches were collected in standard embryo culture medium and carefully cleaned out at about 3 hours of development to remove any unfertilized, dead or deformed embryos. Westerfield, M., The zebrafish book: a guide for the laboratory use of zebrafish (Danio rerio). 1989, Eugene: University of Oregon Press. Between 3 and 5 hours of development, the embryo medium was decanted and the embryos were scooped into 48 well plates (Falcon) containing 300 μl of screening medium (embryo medium plus 1% DMSO, 0.5 M metronidazole, 50 U/ml penicillin, and 50 μg/ml streptomycin) containing pools of small molecules (see chemical section). Approximately 15 embryos were added per well using a chemical weighing spatula. The embryos were then cultured in chemicals overnight at 28.5 degrees C. The crash & burn and standstill phenotypes are first detected by immunostains with cell cycle markersvat 19 hours and 12 hours of development, respectively. By 24 hours of development, there is a strong phenotype for both mutants by immunostains and by morphology. Thus, 24 hours was chosen as the endpoint. The chorions were removed by adding 150 μl of 5 mg/ml pronase (Roche) in embryo medium. After 10 min. in pronase the plate was gently shaken to disrupt the chorions. The screening medium with chemicals and pronase was then pipeted off and 4% paraformaldehyde was added to fix the embryos for whole mount immunostaining.

Chemicals: The chemicals were obtained through a collaboration with the Institute of Chemistry and Cell Biology (ICCB) at Harvard Medical School. The chemical library was the DIVERSet E library (16,320 compounds) purchased from ChemBridge Corporation (San Diego, Calif.). Using a TECAN liquid handling robot, 80 μl of screening medium was transferred to polystyrene 384-well plates (Nunc). These plates were brought to the ICCB where 1 μl of compound (5 mg/ml in DMSO) was robotically pin transferred to the screening medium. The plates were then returned to the TECAN robot which was programmed to aliquot the chemicals to 48 well plates in pools. A matrix pooling strategy was used wherein 16 chemicals are pooled horizontally and vertically, generating 8 pools of 4 letters (see FIG. 3). In the matrix pooling strategy, a chemical is considered a hit only if the phenotype appears in one horizontal pool and one vertical pool. The intersection of the pools in the grid identifies the chemical of interest. Under matrix pooling, each chemical was tested in duplicate. Thus, although there were 15 embryos per well, there were actually 30 embryos per chemical. Given the constraints of plate geometry and the desire to increase throughput, an 8×10 matrix was utilized. The average concentration of each chemical in the pool was 20 μM. A total of 8,000 chemicals were screened in 8 weeks—5,560 chemicals with crash & burn embryos and 2,440 chemicals with standstill embryos.

Whole mount immunostaining: Immunostaining was performed in 48 well grids with a screened bottom. Embryos were transferred from the 48 well plates into the staining grids. Embyos were rinsed in PBS and then incubated for 7 min. in −20 C acetone followed by a water rinse and two rinses in PBST. The embryos were blocked for 30 min at room temperature in PBST plus 5% lamb serum, 10% blocking solution (Roche) and 1% DMSO. A polyclonal antibody to phosphorylated histone H3 (P-H3) (Santa Cruz) was used as a marker for late G2/M phase cells. The embryo grid was incubated overnight at 4 C in primary antibody diluted 1:1000 in block, followed by 3 rinses in PBST. The grid was then transferred to secondary antibody—peroxidase-conjugated goat-anti-rabbit (Jackson Immunochemicals) diluted 1 to 300 in block—for 2 hours at room temperature. After 4 rinses in PBST, diaminobenzidine (DAB, Sigma) at 0.7 mg/ml in PBS was used as a chromogen. The embryos were rinsed in PBS to remove the soluble DAB, and the grid was then transferred to 4% paraformaldehyde. The embryos in paraformaldehyde were transferred to agarose-coated 48 well plates for scoring and storage.

Scoring: The embryos in each well were visually examined for increased or decreased P-H3 staining using a dissecting microscope (Leica). Although the driving force behind the screen was to look for rescue of the mutant phenotype, several other categories of activity were detected or theoretically could be detected, all of which are detailed in the screen results below:

Results

No effect: A chemical was considered to have no effect if 25% of the embryos had a mutant pattern of P-H3 staining and the remaining 75% a wild-type pattern. There was, of course, a normal distribution of mutant embryos centered on 25%. Even if only 1 mutant was present in 30, the chemical was considered to have no effect (unless the mutant exhibited some evidence of partial rescue). As expected, most chemicals had no effect.

Toxic effect: If most of the embryos were dead, delayed, or exhibited some morphologic abnormality, the chemical was considered toxic. Approximately 2% of the compounds were toxic.

Complete rescue: If all embryos had a wild-type phenotype, that chemical was chosen for further analysis. One possibility was that the chemical produced a complete rescue of the mutant phenotype. The other possibility was that there were never any mutants present in the well. With 30 embryos per chemical, the latter possibility can be calculated to occur with a frequency of 0.01%. 12 of 8000 chemicals scored in the “complete rescue” category, but after re-testing with about 100 embryos per chemical, all but one were eliminated. The one chemical (FIGS. 11A-11C) was re-tested with crash & burn embryos again at doses of 9, 12, 16 and 20 μM. At 24 hours of development, no crash & burn mutants were detected in 30-60 embryos at each dose, but a control cohort without chemical exhibited 13 very clear mutants out of 40 (FIGS. 4A-4C). Genotyping of the embryos at the 20 μM dose demonstrated the presence of 12 “mutants” in 56 embryos, despite the lack of a cell cycle phenotype. On the whole, the embryos exhibited a slight developmental delay, but the P-H3 staining was normal, suggesting that the chemical can delay/rescue the crash & burn phenotype without overt toxicity or cell cycle affects on wild-type embryos, an indicator that the chemical may be acting on a specific pathway related to crash & burn. Heterozygotes will be treated with 8G16 to determine if the chemical has chemotherapeutic or chemopreventive activity.

Partial rescue: Partial rescue was considered when mutants were present but the P-H3 staining phenotype was less severe than normally seen. As expected, given the subjective assessment, there were a significant number of false positives in this category. About 20 chemicals where considered as partial rescue candidates, but most were eliminated on re-testing. 8 chemicals (FIG. 11B) were found to partially decrease P-H3 staining in crash & burn embryos, but also decreased staining in wild-type embryos. These

This pattern has been seen with known chemicals that delay the cell cycle in S-phase. For example, if offspring of crash & burn heterozygotes are raised in the presence of aphidicolin, an inhibitor of DNA synthesis, P-H3 staining is decreased in all embryos, including crash & burn (FIG. 4). The chemicals in this category will be further characterized by fluorescence activated cell sorting.

General effects: A chemical that causes cell cycle arrest in any phase would be expected to be identified in this screen. The 8 chemicals already mentioned in the partial rescue category also fall in this general category. In addition, 11 chemicals caused increased P-H3 staining in general. Five of these chemicals were detected in mitotic arrest assays done by other labs using mammalian cells to screen the same chemical library and are not described further here. For the remaining 6 compounds (Group III, FIG. 11C), this activity appears to be novel.

Synthetic lethal: Chemicals that have a synthetic effect would induce a mutant phenotype in heterozygotes but not in wild-types. Such a chemical may or may not have an effect on mutants. Assuming no effect on mutants, 75% of the embryos would have the mutant phenotype (mutants and heterozygotes). Alternatively, if there is an effect on mutants, presumably making the phenotype more severe, 50% of the embryos might have a mutant phenotype and 25% (the homozygous mutants) would have a more severe phenotype. Again, there is a statisical false-positive rate. 7 chemicals scored in the synthetic lethal category, but all were eliminated on re-testing.

Selective toxicity: A chemical could be selectively toxic to the mutants. In that case, the well would contain wild-type embryos and, depending on when death occurred, recognizable dead mutants or fragmented embryonic debris. Such compounds could be retested on larger numbers of embryos and genotyping could be performed to confirm the loss of mutants. No chemicals scored in this category.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

1. A method of screening a test compound for the ability of the compound to alter an inherited phenotype, comprising the steps of:
(a) contacting at least one teleost which has inherited the phenotype with a test compound; and
(b) detecting a change in the inherited phenotype.

2. The method of claim 1, wherein the phenotype is associated with a disease and wherein the disease is selected from the group consisting of cancer, hematologic disease, immunologic disease, angiogenesis, bone diseases, cardiovascular disease, obesity, diabetes, and neurodegenerative disease.

3. The method of claim 1, wherein the teleost is a zebrafish.

4. The method of claim 1, wherein the teleost is a zebrafish embryo.

5. The method of claim 1, wherein the teleost is an embryo, larva or adult.

6. The method of claim 1, wherein the teleost is contained in a microtiter well.

7. The method of claim 1, wherein the test compound is administered to the teleost by dissolving the test compound in media containing the teleost.

8. The method of claim 1, wherein the test compound is administered to the teleost by injecting the test compound into the teleost.

9. The method of claim 1, wherein the test compound is administered to the teleost in conjunction with a carrier.

10. The method of claim 9, wherein the carrier is a solvent, lipid or peptide.

11. The method of claim 1, wherein the test compound is a small molecule, nucleic acid, peptide, protein, glycoprotein, carbohydrate, lipid, or glycolipid.

12. The method of claim 1, wherein the phenotype is characterized by phosphorylated or dephosphorylated cell cycle protein.

13. The method of claim 11, wherein the nucleic acid is DNA or RNA.

14. The method of claim 1, wherein the method comprises screening more than one test compound.

15. A compound obtained by the method of claim 1 or 14.

16. A method of treating a host having a cell cycle defect comprising administering a compound obtained by the method of claim 1 or 14 and a pharmaceutically acceptable carrier.

17. A method of treating a host having a cell cycle defect comprising administering a compound selected from the group consisting of adamantane-1-carboxylic acid (3-hydroxy-pyridin-2-yl)-amide, 4-(4-Allyloxy-3,5-dibromo-benzenesulfonyl)-2,6-dibromo-phenol, 4-Hydroxy-3-[3-(4-hydroxy-phenyl)-acryloyl]-6-methyl-pyran-2-one, 2-Benzoyl-3a,7a-dihydro-indene-1,3-dione, Toluene-4-sulfonic acid 2,4-dinitro-phenyl ester, 3,5-Diiodo-N-[2-chloro-5-(4-chloro-benzenesulfonyl)-phenyl]-2-hydroxy-benzamide, 1-(2-Amino-4-nitro-phenylamino)-3-phenyl-urea, 1-(3,4-Dichloro-phenyl)-2-(2-imino-2H-pyridin-1-yl)-ethanone, 2-(2-o-Tolyloxy-acetylamino)-benzoic acid, N-(2-Chloro-phenyl)-succinamic acid methyl ester, 4-(2-Chloro-5-trifluoromethyl-phenylcarbamoyl)-butyric acid, 4-(Naphthalen-1-ylamino)-3,5-dinitro-benzoic acid, 2-[1-(3-Chloro-phenyl)-2,5-dioxo-pyrrolidin-3-ylsulfanyl]-N-(3-fluoro-phenyl)-acetamide, 2-(5-Hydroxymethyl-8-methyl-3-oxa-bicyclo[3.3.1]non-7-en-2-yl)-phenol, 5-Acetyl-4-(3-hydroxy-phenyl)-6-methyl-3,4-dihydro-1H-pyrimidin-2-one and a pharmaceutically acceptable carrier.

18. An article of manufacture comprising packaging material and a pharmaceutical composition contained within said packaging material, wherein said packaging material comprises a label which indicates said pharmaceutical may be administered, for a sufficient term at an effective dose, for treating and/or preventing cancer, hematologic disease, immunologic disease, angiogenesis, bone diseases, cardiovascular disease, obesity, diabetes, and neurodegenerative disease in a mammal, wherein the pharmaceutical composition comprises a compound obtained by the method of claim 1 or 14 together with a pharmaceutically acceptable carrier.

19. An article of manufacture comprising packaging material and a pharmaceutical composition contained within said packaging material, wherein said packaging material comprises a label which indicates said pharmaceutical may be administered, for a sufficient term at an effective dose, for treating and/or preventing cancer, hematologic disease, immunologic disease, angiogenesis, bone diseases, cardiovascular disease, obesity, diabetes, and neurodegenerative disease in a mammal, wherein the pharmaceutical composition comprises a compound selected from a group consisting of adamantane-1-carboxylic acid (3-hydroxy-pyridin-2-yl)-amide, 4-(4-Allyloxy-3,5-dibromo-benzenesulfonyl)-2,6-dibromo-phenol, 4-Hydroxy-3-[3-(4-hydroxy-phenyl)-acryloyl]-6-methyl-pyran-2-one, 2-Benzoyl-3a,7a-dihydro-indene-1,3-dione, Toluene-4-sulfonic acid 2,4-dinitro-phenyl ester, 3,5-Diiodo-N-[2-chloro-5-(4-chloro-benzenesulfonyl)-phenyl]-2-hydroxy-benzamide, 1-(2-Amino-4-nitro-phenylamino)-3-phenyl-urea, 1-(3,4-Dichloro-phenyl)-2-(2-imino-2H-pyridin-1-yl)-ethanone, 2-(2-o-Tolyloxy-acetylamino)-benzoic acid, N-(2-Chloro-phenyl)-succinarnic acid methyl ester, 4-(2-Chloro-5-trifluoromethyl-phenylcarbamoyl)-butyric acid, 4-(Naphthalen-1-ylamino)-3,5-dinitro-benzoic acid, 2-[1-(3-Chloro-phenyl)-2,5-dioxo-pyrrolidin-3-ylsulfanyl]-N-(3-fluoro-phenyl)-acetamide, 2-(5-Hydroxymethyl-8-methyl-3-oxa-bicyclo[3.3.1]non-7-en-2-yl)-phenol, 5-Acetyl-4-(3-hydroxy-phenyl)-6-methyl-3,4-dihydro-1H-pyrimidin-2-one together with a pharmaceutically acceptable carrier.
